SEQUENCE LISTING (80 sequences)

SEQ ID NO: 1 - protein, 1157 aa, synthetic construct
  REGION 1..1157: note = peptide
MEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRARMALPVTALLLPLALLLHAARPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKFVPVFLPAKPTTTPAPRPPTPAPTIASQPLSLRPEACRPAAGGAVHTRGLDFACDPFFFCCFIAVAMGIRFIIMVAWRRKAKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGGGSHHHHHH

SEQ ID NO: 2 - protein, 11 aa, synthetic construct
  REGION 1..11: note = peptide
YGRKKRRQRRR

SEQ ID NO: 3 - protein, 9 aa, synthetic construct
  REGION 1..9: note = peptide
RKKRRQRRR

SEQ ID NO: 4 - protein, 6 aa, synthetic construct
  REGION 1..6: note = peptide
RRRRRR

SEQ ID NO: 5 - protein, 8 aa, synthetic construct
  REGION 1..8: note = peptide
RRRRRRRR

SEQ ID NO: 6 - protein, 9 aa, synthetic construct
  REGION 1..9: note = peptide
RRRRRRRRR

SEQ ID NO: 7 - protein, 14 aa, synthetic construct
  REGION 1..14: note = peptide
RQIKIWFQNRRMKW

SEQ ID NO: 8 - protein, 16 aa, synthetic construct
  REGION 1..16: note = peptide
RQIKIWFQNRRMKWKK

SEQ ID NO: 9 - protein, 18 aa, synthetic construct
  REGION 1..18: note = peptide
VRLPPPVRLPPPVRLPPP

SEQ ID NO: 10 - protein, 12 aa, synthetic construct
  REGION 1..12: note = peptide
RRQRRTSKLMKR

SEQ ID NO: 11 - protein, 16 aa, synthetic construct
  REGION 1..16: note = peptide
AAVALLPAVLLALLAP

SEQ ID NO: 12 - protein, 17 aa, synthetic construct
  REGION 1..17: note = peptide
TRQARRNRRRRWRERQR

SEQ ID NO: 13 - protein, 15 aa, synthetic construct
  REGION 1..15: note = peptide
RRRRNRTRRNRRRVR

SEQ ID NO: 14 - protein, 13 aa, synthetic construct
  REGION 1..13: note = peptide
TRRQRTRRATTNR

SEQ ID NO: 15 - protein, 16 aa, synthetic construct
  REGION 1..16: note = peptide
KRPAAIKKAGQAKKKK

SEQ ID NO: 16 - protein, 27 aa, synthetic construct
  REGION 1..27: note = peptide
GWTLNSAGYLLGKINLKALAALAKKIL

SEQ ID NO: 17 - protein, 18 aa, synthetic construct
  REGION 1..18: note = peptide
LLIILRRRIRKQAHAHSK

SEQ ID NO: 18 - protein, 14 aa, synthetic construct
  REGION 1..14: note = peptide
YGRKKRRQRRRPPQ

SEQ ID NO: 19 - protein, 16 aa, synthetic construct
  REGION 1..16: note = peptide
RQIKIWFQNRRMKWKK

SEQ ID NO: 20 - protein, 26 aa, synthetic construct
  REGION 1..26: note = peptide
GWTLNSAGYLLGKINLKALAALAKKL

SEQ ID NO: 21 - protein, 7 aa, synthetic construct
  REGION 1..7: note = peptide
LCLRPVG

SEQ ID NO: 22 - protein, 10 aa, synthetic construct
  REGION 1..10: note = peptide
RRRRRRRRRR

SEQ ID NO: 23 - protein, 10 aa, synthetic construct
  REGION 1..10: note = peptide
KKKKKKKKKK

SEQ ID NO: 24 - protein, 18 aa, synthetic construct
  REGION 1..18: note = peptide
KLALKLALKALKAALKLA

SEQ ID NO: 25 - protein, 21 aa, synthetic construct
  REGION 1..21: note = peptide
KETWWETWWTEWSQPKKKRKV

SEQ ID NO: 26 - protein, 12 aa, synthetic construct
  REGION 1..12: note = peptide
PLILLRLLRGQF

SEQ ID NO: 27 - protein, 12 aa, synthetic construct
  REGION 1..12: note = peptide
PLIYLRLLRGQF

SEQ ID NO: 28 - protein, 15 aa, synthetic construct
  REGION 1..15: note = peptide
KLWMRWYSPTTRRYG

SEQ ID NO: 29 - protein, 18 aa, synthetic construct
  REGION 1..18: note = peptide
KLALKLALKALKAALKLA

SEQ ID NO: 30 - protein, 18 aa, synthetic construct
  REGION 1..18: note = peptide
LLIILRRRIRKQAHAHSK

SEQ ID NO: 31 - protein, 10 aa, synthetic construct
  REGION 1..10: note = peptide
RRIPNRRPRR

SEQ ID NO: 32 - protein, 12 aa, synthetic construct
  REGION 1..12: note = peptide
RRQRRTSKLMKR

SEQ ID NO: 33 - protein, 4 aa, synthetic construct
  REGION 1..4: note = peptide
GGFG

SEQ ID NO: 34 - protein, 4 aa, synthetic construct
  REGION 1..4: note = peptide
RRAR

SEQ ID NO: 35 - protein, 4 aa, synthetic construct
  REGION 1..4: note = peptide
GFLG

SEQ ID NO: 36 - protein, 4 aa, synthetic construct
  REGION 1..4: note = peptide
ALAL

SEQ ID NO: 37 - protein, 21 aa, synthetic construct
  REGION 1..21: note = peptide
MALPVTALLLPLALLLHAARP

SEQ ID NO: 38 - protein, 20 aa, synthetic construct
  REGION 1..20: note = peptide
HMIGICVTLTVIIVCSVFIY

SEQ ID NO: 39 - protein, 21 aa, synthetic construct
  REGION 1..21: note = peptide
LLLLFWLGWLGMLAGAVVIIV

SEQ ID NO: 40 - protein, 24 aa, synthetic construct
  REGION 1..24: note = peptide
ALSPVELGLLLLPFVVMLLAALCV

SEQ ID NO: 41 - protein, 21 aa, synthetic construct
  REGION 1..21: note = peptide
IMIVLVMLLNIGLAILFVHFL

SEQ ID NO: 42 - protein, 21 aa, synthetic construct
  REGION 1..21: note = peptide
STVFTVLLLLLGMAAYSFGWV

SEQ ID NO: 43 - protein, 21 aa, synthetic construct
  REGION 1..21: note = peptide
GLLACIAAVLMLPAFLYLHYV

SEQ ID NO: 44 - protein, 42 aa, synthetic construct
  REGION 1..42: note = peptide
TYVMAAVLCQVIIFGCMFEIVTPPIVSTGMALLLILYLLFYM

SEQ ID NO: 45 - protein, 21 aa, synthetic construct
  REGION 1..21: note = peptide
IGWMLSFTISELLFLIILAAI

SEQ ID NO: 46 - protein, 20 aa, synthetic construct
  REGION 1..20: note = peptide
VGHSLSIFTLVISLGIFVFF

SEQ ID NO: 47 - protein, 20 aa, synthetic construct
  REGION 1..20: note = peptide
VTLHKNMFLTYILNSMIIII

SEQ ID NO: 48 - protein, 23 aa, synthetic construct
  REGION 1..23: note = peptide
ILHFFHQYMMACNYFWMLCEGIY

SEQ ID NO: 49 - protein, 20 aa, synthetic construct
  REGION 1..20: note = peptide
WYYLLGWGFPLVPTTIHAIT

SEQ ID NO: 50 - protein, 24 aa, synthetic construct
  REGION 1..24: note = peptide
LLYIIHGPVMAALVVNFFFLLNIV

SEQ ID NO: 51 - protein, 18 aa, synthetic construct
  REGION 1..18: note = peptide
ATMILVPLLGIQFVVFPW

SEQ ID NO: 52 - protein, 22 aa, synthetic construct
  REGION 1..22: note = peptide
YVMHSLIHFQGFFVATIYCFCN

SEQ ID NO: 53 - protein, 21 aa, synthetic construct
  REGION 1..21: note = peptide
AAVFFGCTFVAFGPAFALFLI

SEQ ID NO: 54 - protein, 21 aa, synthetic construct
  REGION 1..21: note = peptide
VIILVAGAFFWLVSLLLASVV

SEQ ID NO: 55 - protein, 21 aa, synthetic construct
  REGION 1..21: note = peptide
YGLLIFGAAVSVLLQEVFRFA

SEQ ID NO: 56 - protein, 21 aa, synthetic construct
  REGION 1..21: note = peptide
YVSGLSFGIISGVFSVINILA

SEQ ID NO: 57 - protein, 21 aa, synthetic construct
  REGION 1..21: note = peptide
TSAFLTAAIILLHTFWGVVFF

SEQ ID NO: 58 - protein, 18 aa, synthetic construct
  REGION 1..18: note = peptide
ATMILVPLLGIQFVVFPW

SEQ ID NO: 59 - protein, 21 aa, synthetic construct
  REGION 1..21: note = peptide
LLPIYAVTVSMGLWAFITAGG

SEQ ID NO: 60 - protein, 21 aa, synthetic construct
  REGION 1..21: note = peptide
NIWTHLLGCVFFLCLGIFYMF

SEQ ID NO: 61 - protein, 21 aa, synthetic construct
  REGION 1..21: note = peptide
VVFGLFFLGAILCLSFSWLFH

SEQ ID NO: 62 - protein, 21 aa, synthetic construct
  REGION 1..21: note = peptide
LFSKLDYSGIALLIMGSFVPW

SEQ ID NO: 63 - protein, 21 aa, synthetic construct
  REGION 1..21: note = peptide
CFIYLIVICVLGIAAIIVSQW

SEQ ID NO: 64 - protein, 21 aa, synthetic construct
  REGION 1..21: note = peptide
YRGVRAGVFLGLGLSGIIPTL

SEQ ID NO: 65 - protein, 21 aa, synthetic construct
  REGION 1..21: note = peptide
QIGWLMLMASLYITGAALYAA

SEQ ID NO: 66 - protein, 21 aa, synthetic construct
  REGION 1..21: note = peptide
QLFHIFVVAGAFVHFHGVSNL

SEQ ID NO: 67 - protein, 448 aa, synthetic construct
  REGION 1..448: note = peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 55 aa, synthetic construct
  REGION 1..55: note = peptide
FVPVFLPAKPTTTPAPRPPTPAPTIASQPLSLRPEACRPAAGGAVHTRGLDFACD

SEQ ID NO: 69 - protein, 5 aa, synthetic construct
  REGION 1..5: note = peptide
GGGGS

SEQ ID NO: 70 - protein, 5 aa, synthetic construct
  REGION 1..5: note = peptide
GGGSS

SEQ ID NO: 71 - protein, 5 aa, synthetic construct
  REGION 1..5: note = peptide
GGGSG

SEQ ID NO: 72 - protein, 10 aa, synthetic construct
  REGION 1..10: note = peptide
GGGGSGGGGS

SEQ ID NO: 73 - protein, 6 aa, synthetic construct
  REGION 1..6: note = peptide
HHHHHH

SEQ ID NO: 74 - protein, 8 aa, synthetic construct
  REGION 1..8: note = peptide
HHHHHHHH

SEQ ID NO: 75 - protein, 10 aa, synthetic construct
  REGION 1..10: note = peptide
HHHHHHHHHH

SEQ ID NO: 76 - protein, 218 aa, synthetic construct
  REGION 1..218: note = peptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 77 - protein, 396 aa, synthetic construct
  REGION 1..396: note = peptide
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 78 - protein, 8 aa, synthetic construct
  REGION 1..8: note = peptide
WSHPQFEK

SEQ ID NO: 79 - protein, 15 aa, synthetic construct
  REGION 1..15: note = peptide
GGGGSGGGGSGGGGS

SEQ ID NO: 80 - protein, 1046 aa, synthetic construct
MDIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIKGGGGSGGGSGGGGSGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPRRARSMALPVTALLLPLALLLHAARPDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGSGGGGSGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSESKYGPPCPPCPFWVLVVVGGVLACYSLLVTVAFIIFWVWRRKAKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGGGSHHHHHH

(19) United States
(12) Patent Application Publication
(10) Pub. No.: US 2025/0090662 A1
(43) Pub. Date: Mar. 20, 2025

(54) COMPOSITION AND METHOD OF USE RECOMBINANT FUSION PROTEIN TO GENERATE CAR-IMMUNE CELLS

(71) Applicant: SINGULAR IMMUNE, INC., Dallas, TX (US)

(72) Inventors: Youngwook WON, Southlake, TX (US); Seungmin HAN, Denton, TX (US); David BULL, Salt Lake City, UT (US)

(73) Assignee: SINGULAR IMMUNE, INC., Dallas, TX (US)

(21) Appl. No.: 18/884,392

(22) Filed: Sep. 13, 2024

Related U.S. Application Data
(60) Provisional application No. 63/583,013, filed on Sep. 15, 2023.

(51) Int. Cl. A61K 39/00; C07K 14/725; C07K 16/28; C07K 16/30

(52) U.S. Cl. CPC A61K 40/4202; A61K 40/11; A61K 40/15; A61K 40/31; A61K 40/4211; C07K 14/7051; C07K 16/2803; C07K 16/2827; C07K 16/30; C07K 2317/622

(57) ABSTRACT

Provided herein are a recombinant chimeric antigen receptor (CAR) fusion protein, a method of modifying an immune cell into a CAR immune cell by treating the immune cell with the recombinant CAR fusion protein, and a method of treating cancer by administering the CAR immune cell to a subject in need thereof.

### CROSS REFERENCE TO RELATED PATENT APPLICATION

This application claims priority to U.S. Provisional Application No. 63/583,013 filed Sep. 15, 2023, the entire disclosure of which is incorporated herein by reference.

### REFERENCE TO SEQUENCE LISTING

The Sequence Listing submitted as a XML file named “A302014_sequence listing as filed”, created on Sep. 12, 2024, and having a size of 102,789 bytes is hereby incorporated by reference.

### FIELD

Provided herein are a recombinant chimeric antigen receptor (CAR) fusion protein, a method of modifying an immune cell into a CAR immune cell by treating the immune cell with the recombinant CAR fusion protein, and a method of treating cancer by administering the CAR immune cell to a subject in need thereof.

### BACKGROUND

Chimeric antigen receptor (CAR) technology has developed to modify immune cells into cancer-specifically stimulated immune cells. CAR-T or CAR-NK cells are widely evaluated in various clinical trials and few of them are being prescribed in clinics. A CAR is composed of an extracellular scFv domain for cancer targeting, intracellular co-stimulatory domains, and transmembrane domains. CAR-immune cells can recognize cancer cells through the binding of the extracellular scFv antibody to the target antigen on cancer cells, which in turn leads to the stimulation of the immune cells to exert stronger anti-cancer activity. The conventional CAR technology raises limitations in its use due to the use of viral vectors for CAR-gene transfer, lengthy generation time that makes it not a feasible option for patients who need immediate treatment, and more importantly, the need for isolation, modification, expansion ex vivo prior to infusion back to the patient.

### SUMMARY

To overcome the above limitations, the present disclosure provides a novel recombinant CAR fusion protein capable of modifying immune cells into CAR-immune cells without the need for genetic modifications.

In particular, disclosed is a recombinant chimeric antigen receptor (CAR) fusion protein comprising an immune cell targeting domain, a cleavable peptide, a membrane targeting domain, a cancer targeting domain, a transmembrane domain, and an intracellular signaling domain. The above recombinant CAR fusion protein includes, in any order, the immune cell targeting domain, the cleavable peptide, the membrane targeting domain, the cancer targeting domain, the transmembrane domain, and the intracellular signaling domain.

In one embodiment, the immune cell targeting domain is a NK cell targeting domain, a T cell targeting domain, a dendritic cell targeting domain, a macrophage targeting domain, a peripheral blood mononuclear cell targeting domain, or a B cell targeting domain. In another embodiment, the immune cell targeting domain is an antibody or peptide specifically binding to an immune cell, and comprising one or more of scFv, bispecific scFv, V_{HH}, V_{H}, V_{L}, F_{ab}, CH1, CH2, CH3, or CL. In one embodiment, the cleavable peptide is a peptide which is configured to be enzymatically cleaved. In one embodiment, the membrane targeting domain is a peptide from secretory protein, membrane receptor, surface protein, human oncostatin M, VSV-G, BM40, secrecon, human Ig kappa, human Ig heavy, tPA, human chymotrypsinogen, human trysinogen-2, human IL-2, guassia luciferase, human serum albumin, influenza haemagglutinin, human insulin, silkworm fibroin LC, CD33, CD8, interleukin-1 receptor type 1, 4F2 cell-surface antigen heavy chain, a linker for activation of T-cells family member 1, junctophilin-1, antilisterial bacteriocin subtilosin biosynthesis protein AlbG, calcitonin receptor, gamma-secretase subunit APH-1A, or adipnectin receptor protein 2. In one embodiment, the cancer targeting domain is an antibody or peptide specifically binding to a cancer cell, and comprising one or more of scFv, bispecific scFv, V_{HH}, V_{H}, V_{L}, F_{ab}, CH1, CH2, CH3, or CL. In another embodiment, the recombinant CAR fusion protein further comprises a hinge region between the cancer targeting domain and the transmembrane domain. In one embodiment, the transmembrane domain is CD28, CD3, CD4, CD7, CD8, FcεR1γ, ICOS, H2-Kb, NKG2D, CD16, NKp44, or NKp46. In one embodiment, the intracellular signaling domain comprises one or more of CD3 zeta (CD35), 2B4, DAP10, DAP12, GIRT, CD137, OX40, 41BB, CD27, CD28, 2B4, CD137, CD40, and KIR2DS2. In one embodiment, the recombinant CAR fusion protein further comprises a tag sequence. In another embodiment, the tag sequence is selected from the group consisting of a metal affinity tag, charge-based tag, epitope peptides, protein-affinity tag, streptavidin/biotin-based tag, histidine tag, glutathione-S-transferase tag, or hemagglutinin tag.

The present disclosure also provides a method of modifying an immune cell into a chimeric antigen receptor (CAR) immune cell, comprising treating the immune cell with the recombinant CAR fusion protein discussed above. In one embodiment, the CAR fusion protein is in a concentration of about 100 nM to about 2,000 nM.

The present disclosure also provides a method of treating cancer comprising administering a CAR immune cell, which has been prepared by treating an immune cell with the a recombinant CAR fusion protein, to a subject in need thereof.

The present disclosure further provides a method of treating cancer comprising administering the recombinant CAR fusion protein discussed above to a subject in need thereof. In one embodiment, the CAR fusion protein is in a concentration of about 100 nM to about 2,000 nM.

The present disclosure also provides a method of treating cancer comprising administering to a subject in need thereof: (i) a recombinant chimeric antigen receptor (CAR) fusion protein comprising an immune cell targeting domain, a cleavable peptide, a membrane targeting domain, a cancer targeting domain, a transmembrane domain, and an intracellular signaling domain; and (ii) a CAR immune cell. In one embodiment, the CAR immune cell is prepared by treating an immune cell with the recombinant CAR fusion protein.

Additional embodiments are described herein.

### BRIEF DESCRIPTION OF THE DRAWINGS

FIG. 1 shows the amino acid sequence of the recombinant CAR fusion protein against targeting PD-L1.

FIG. 2 shows the structure of the recombinant CAR fusion protein of the present disclosure and describes how it modifies immune cells to chimeric antigen receptor (CAR) immune cells.

FIG. 3 shows SDS-PAGE and Western blot results.

FIG. 4 shows FACS data confirming the uptake of the recombinant CAR fusion protein against anti-PD-L1 by NK cells.

FIGS. 5A and 5B show FACS data comparing the uptake efficiency of the recombinant CAR fusion protein against anti-PD-L1 by NK cells at various concentrations.

FIG. 6 shows FACS data demonstrating that the CAR-NK cells had higher PE intensity, which indicated that more PD-L1 protein was bound to the CAR-NK cells due to the CAR protein.

FIG. 7 shows data comparing cytotoxic/cytolytic cytokine secretion upon PD-L1 binding when the CAR-NK cells and the NK cells were treated.

FIG. 8 shows data comparing the anti-cancer activity of the NK cells and the CAR-NK cells.

FIG. 9 shows data comparing cytokine secretion when the CAR-NK cells and the NK cells were treated.

FIGS. 10A, 10B and 10C show data relating to in vivo CAR-NK retention in vitro.

FIG. 11 shows in vivo tumor inhibition by the in vivo CAR protein.

FIG. 12 shows data relating to human cytokine in tumor tissues and plasma.

FIG. 13 shows data relating to in vivo CAR-Modification on blood human NK and human T cells.

FIG. 14 shows data confirming that NK cells become more cytolytic upon in vivo CAR injection.

FIG. 15 shows data relating to initial Liver toxicity test.

FIGS. 16A, 16B and 16C show data confirming that in vivo CAR protein generates a comprehensive Immune response in the TME.

FIG. 17 shows data relating to initial immunogenicity test.

FIG. 18 shows data relating to in vivo CAR-NK biodistribution.

FIG. 19 shows the amino acid sequence of the recombinant CAR fusion protein against targeting TROP2.

FIGS. 20A, 20B and 20C show data confirming that uptake of the recombinant CAR fusion protein against anti-TROP2 specifically by NK cells and T cells among peripheral blood mononuclear cell (PBMC).

FIG. 21 shows data comparing the anti-cancer activity of PBMC and recombinant CAR fusion protein-treated PBMC against TROP2-positive cancer cell.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide” is a reference to one or more peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting growth and/or spread of the cancer cells, killing the cancer cells, or shrinking the cancer cells. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab) 2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell or NK cell that specifically binds with a cognate stimulatory ligand present on an antigen presenting cell.

A “stimulatory ligand,” as used herein, means a ligand that when present on an antigen presenting cell (e.g., an a APC, a dendritic cell, a B-cell, and the like) can specifically bind with a cognate binding partner (referred to herein as a “stimulatory molecule”) on a T cell or NK cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, a superagonist anti-CD2 antibody and the like.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to kidney cancer, spleen cancer, lung cancer, liver cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “therapeutically effective amount” means an amount of a therapeutic, prophylactic, and/or diagnostic agent (e.g., CAR) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

### DETAILED DESCRIPTION

### 1. The Recombinant Chimeric Antigen Receptor (CAR) Fusion Protein

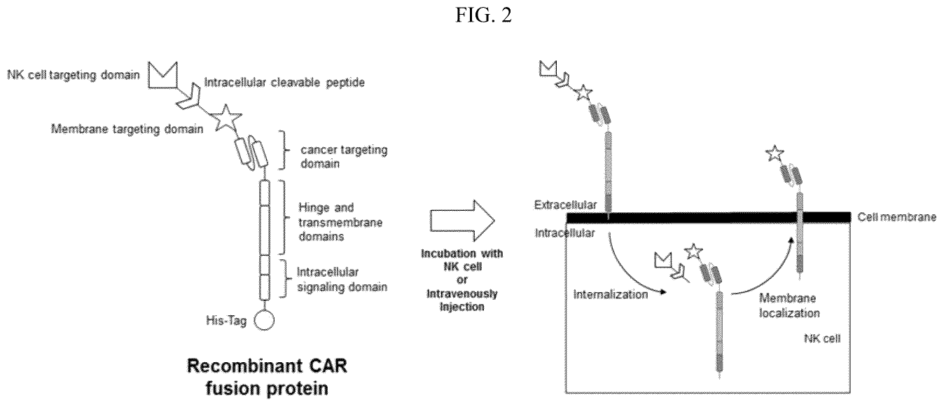

The present disclosure provides a recombinant chimeric antigen receptor (CAR) fusion protein comprising an immune cell targeting domain, a cleavable peptide, a membrane targeting domain, a cancer targeting domain, a transmembrane domain, and an intracellular signaling domain. The structure of an exemplary embodiment of the recombinant CAR fusion protein of the present disclosure is described in FIG. 2. In this embodiment, the recombinant CAR fusion protein was designed to modify NK cells into CAR NK cells via in vivo application. As described in FIG. 2, the recombinant CAR fusion protein can target NK cells by the NK cell targeting domain (i.e., the immune cell targeting domain), and enter into NK cell's cytoplasm by antigen-antibody binding. Once the recombinant CAR fusion protein is entered into the cytoplasm, the recombinant CAR fusion protein is separated into the NK cell targeting domain and the rest of the recombinant CAR fusion protein domains because of the cleavable peptide (which is enzymatically cleaved). Then, the membrane targeting domain induces the rest of the recombinant CAR fusion protein domains (i.e., the membrane targeting domain, cancer targeting domain, transmembrane domain, and intracellular signaling domain; also referred to the CAR domain) locate in the cell membrane. Therefore, the CAR domain can increase anti-cancer activity of the NK cells upon binding with cancer cells.

The recombinant CAR fusion protein of the present disclosure may comprise, in any order, an immune cell targeting domain, a cleavable peptide, a membrane targeting domain, a cancer targeting domain, a transmembrane domain, and an intracellular signaling domain.

In one embodiment, the recombinant CAR fusion protein of the present disclosure may comprise, in order, an intracellular signaling domain, a transmembrane domain, a cancer targeting domain, a membrane targeting domain, a cleavable peptide, and an immune cell targeting domain.

In another embodiment, the recombinant CAR fusion protein of the present disclosure may comprise, in order, an immune cell targeting domain, a cleavable peptide, a membrane targeting domain, a cancer targeting domain, a transmembrane domain, and an intracellular signaling domain.

The recombinant CAR fusion protein of the present disclosure has advantages over the conventional CAR technology as it can generate CAR-immune cells without viral vector, within a day, and either ex vivo or in vivo. More importantly, this technology allows a cell-free immunotherapy, which is safer and less expensive than the conventional CAR-immune cells.

### The Immune Cell Targeting Domain

In one embodiment, the immune cell targeting domain identifies the target immune cell, and thus the recombinant CAR fusion protein enters into the target immune cell through antigen-antibody binding. The immune cell targeting domain may be an immune cell capturing domain, an immune cell binding domain, an anti-immunoreceptor domain, an immunoreceptor binding domain, or an immunoreceptor targeting domain. The immune cell targeting domain is a NK cell targeting domain, a T cell targeting domain, a dendritic cell targeting domain, a macrophage targeting domain, a peripheral blood mononuclear cell targeting domain, or a B cell targeting domain.

For instance, the NK cell targeting domain may be included in the recombinant CAR fusion protein such that the recombinant CAR fusion protein identifies and enters into NK cells. The NK cell targeting domain may be an antibody, antibody fragment or an antigen-binding fragment targeting NK cells. For instance, the NK cell targeting domain may be a NK cell-targeting antibody, NK cell-targeting-scFv antibody, NK cell-targeting bispecific scFv antibody, NK cell-targeting nanobody, NK cell-targeting camel antibody, or a peptide comprising a NK cell-targeting antibody-V_{H}, V_{L}, V_{HH}, hinge, CH1, CH2, CH3, or CL. The NK cell targeting antibody can be anti-NKp30, anti-NKp44, anti-NKp46, anti-NKp80, anti-NKG2A, anti-NKG2C, anti-NKG2D, anti-CD16, anti-CD56, anti-KIR-s, anti-CD122, anti-PD-1, anti-TIGIT, anti-LAG3, or anti-KLRG.

In addition, the T cell targeting domain may be included in the recombinant CAR fusion protein so that the recombinant CAR fusion protein identifies and enters into T cells. For instance, the T cell targeting domain may be a T cell-targeting antibody, T cell-targeting-scFv antibody, T cell targeting bispecific scFv antibody, T cell-targeting nanobody, T cell-targeting camel antibody, a peptide comprising a T cell-targeting antibody-V_{H}, hinge, CH1, CH2, CH3, or CL. The T cell targeting antibody can be anti-CD3, anti-CD8, anti-CD4, anti-CD45R, anti-CD40, anti-CD25, anti-OX40, anti-NKG2D, anti-NKp46, anti-NKp30, anti-PD-1, anti-TIM3, anti-TIGIT, anti-LAG3, or anti-CTLA4.

In another embodiment, when the recombinant CAR fusion protein is used to modify an immune cell into a chimeric antigen receptor (CAR) immune cell in ex vivo application, since specific immune cells may be directly treated with the recombinant CAR fusion protein, the immune cell targeting domain may be also selected from any protein-transduction domain, which is not specific to the target immune cell, but allows non-specific transduction of the recombinant CAR fusion protein, into any cells. In one embodiment, the immune cell targeting domain for ex vivo application (which is not specific to certain immune cells) may include Tat or a Tat peptide, poly-arginine, antennapedia (Antp) or Antp peptide, penetratin, SAP, PTD-5, K-FGF (SN50 peptide), HIV-1 Rev, FHV, HTLV-II, NLS, transportan, pVEC. Examples of these domains can be, but are not limited to Tat (YGRKKRRQRRR; SEQ ID NO: 2) or a Tat peptide (RKKRRQRRR; SEQ ID NO: 3), poly-arginine (RRRRRR; SEQ ID NO: 4; RRRRRRRR; SEQ ID NO: 5; RRRRRRRRR; SEQ ID NO: 6), antennapedia (Antp) or Antp peptide (RQIKIWFQNRRMKW; SEQ ID NO: 7), penetratin (RQIKIWFQNRRMKWKK; SEQ ID NO: 8), SAP (VRLPPPVRLPPPVRLPPP; SEQ ID NO: 9), PTD-5 (RRQRRTSKLMKR; SEQ ID NO: 10), K-FGF (SN50 peptide) (AAVALLPAVLLALLAP; SEQ ID NO: 11), HIV-1 Rev (TRQARRNRRRRWRERQR; SEQ ID NO: 12), FHV (RRRRNRTRRNRRRVR; SEQ ID NO: 13), HTLV-II (TRRQRTRRATTNR; SEQ ID NO: 14), NLS (KRPAAIKKAGQAKKKK; SEQ ID NO: 15), transportan (GWTLNSAGYLLGKINLKALAALAKKIL; SEQ ID NO: 16), pVEC (LLIILRRRIRKQAHAHSK; SEQ ID NO: 17). See also Table 1 below.

TABLE 1

| Name | Sequence |
|---|---|
| Tat 47-60 | YGRKKRRQRRRPPQ |
| | (SEQ ID NO: 18) |
| Penetratin | RQIKIWFQNRRMKWKK |
| | (SEQ ID NO: 19) |
| Transportan ¹ | GWTLNSAGYLLGKINLKALAALAKKL |
| | (SEQ ID NO: 20) |
| Xentry, N-terminal | LCLRPVG (SEQ ID NO: 21) |
| region of the | |
| X-protein of the | |
| hepatitis B virus | |
| Poly Arginine₈₋₁₀ | RRRRRRRR(RR) (SEQ ID NO: 22) |
| Lysine₈₋₁₀ ² | KKKKKKKK(KK) (SEQ ID NO: 23) |
| MAP ³ | KLALKLALKALKAALKLA SEQ ID |
| | NO: 24) |
| Pep-1 ⁴ | KETWWETWWTEWSQPKKKRKV |
| | (SEQ ID NO: 25) |
| Pept1 ⁵ | PLILLRLLRGQF (SEQ ID NO: 26) |
| Pept2 ⁵ | PLIYLRLLRGQF (SEQ ID NO: 27) |
| IVV-14 ⁶ | KLWMRWYSPTTRRYG |
| | (SEQ ID NO: 28) |
| Amphiphilic model | KLALKLALKALKAALKLA (SEQ ID |
| peptide ⁷ | NO: 29) |
| pVEC ⁸ | LLIILRRRIRKQAHAHSK |
| | (SEQ ID NO: 30) |
| HRSV ⁹ | RRIPNRRPRR (SEQ ID NO: 31) |
| PTD-5 ¹⁰ | RRQRRTSKLMKR (SEQ ID NO: 32) |

¹ Pooga et al., FASEB J. 12: 67-77 (1998)
² Mi et al., J. Biol. Chem. 277(33): 30208-30218 (2002)
³ Robbins et al., Cancer Res. 51: 3657-3662 (1991)
⁴ Deshayes et al., Biochemistry 43(6): 1449-1457 (2004)
⁵ Marks et al., J. Am. Chem. Soc. 133(23): 8995-9004 (2011)
⁶ Kamide et al., Int. J. Mol. Med. 25(1): 41-51 (2010)
⁷ Lindgren et al. Trend Pharmacol. Sci. 21(3): 99-103 (2000)
⁸ Sidhu and Weiss, in Anticancer Drug Development, Baguley and Kerr, Ed., Academic Press 237-248 (2002)
⁹ Langedijk et al, in Drug Transport(ers) and the Diseased Brain, International Congress Series, Elsevier 95-107 (2005)
¹⁰ Mi et al., Mol. Ther. 2(4): 339-347 (2000)

### The Cleavable Peptide

In one embodiment, the cleavable peptide is enzymatically cleaved to separate the immune cell targeting domain from the rest of the recombinant CAR fusion protein domains once the recombinant CAR fusion protein enters into the immune cell.

The cleavable peptide may be a cleavable site, or a cleavable domain.

The cleavable peptide may be protease cleavage site: Gly-Gly-Phe-Gly (GGFG; SEQ ID NO: 33), Furin cleavage site (RRAR; SEQ ID NO: 34), Cathepsin cleavage site (Phe-Lys (FK), Ala-Ala-Asn (AAN), Gly-Phe-Leu-Gly (GFLG; SEQ ID NO: 35)), or Legumain cleavage site (Ala-Leu-Ala-Leu (ALAL; SEQ ID NO: 36)).

### The Membrane Targeting Domain

In one embodiment, once the immune cell targeting domain is separated from the rest of the recombinant CAR fusion protein domains by the cleavable peptide is enzymatically cleaved, the membrane targeting domain induces the rest of the recombinant CAR fusion protein domains to locate in cell membrane (triggers translocation of the rest of the recombinant CAR fusion protein domains toward the outer cell membrane; plasma membrane).

The membrane targeting domain may be a secretory domain.

The membrane targeting domain may be a peptide from T-cell surface glycoprotein CD8 alpha chain (MALPVTALLLPLALLLHAARP; SEQ ID NO: 37), interleukin-1 receptor type 1, 4F2 cell-surface antigen heavy chain, linker for activation of T-cells family member 1, junctophilin-1, antilisterial bacteriocin subtilosin biosynthesis protein AlbG, calcitonin receptor, gamma-secretase subunit APH-1A, adipnectin receptor protein 2. Examples of these peptides can be, but are not limited to, interleukin-1 receptor type 1 (HMIGICVTLTVIIVCSVFIY; SEQ ID NO: 38), 4F2 cell-surface antigen heavy chain (LLLLFWLGWLGMLAGAVVIIV; SEQ ID NO: 39), linker for activation of T-cells family member 1 (ALSPVELGLLLLPFVVMLLAALCV; SEQ ID NO: 40), junctophilin-1 (IMIVLVMLLNIGLAILFVHFL; SEQ ID NO: 41), antilisterial bacteriocin subtilosin biosynthesis protein AlbG (STVFTVLLLLLGMAAYSFGWV-SEQ ID NO: 42; GLLACIAAVLMLPAFLYLHYV-SEQ ID NO: 43; TYVMAAVLCQVIIFGCMFEIV; TPPIVSTGMALLLILYLLFYM-SEQ ID NO: 44; IGWMLSFTISELLFLIILAAI-SEQ ID NO: 45), calcitonin receptor (VGHSLSIFTL VISLGIFVFF-SEQ ID NO: 46; VTLHKNMFLTYILNSMIIII-SEQ ID NO: 47; ILHFFHQYMMACNYFWMLCEGIY-SEQ ID NO: 48; WYYLLGWGFPLVPTTIHAIT-SEQ ID NO: 49; LLYIIHGPVMAALVVNFFFLLNIV-SEQ ID NO: 50; ATMILVPLLGIQFVVFPW-SEQ ID NO: 51; YVMHSLIHFQGFFVATIYCFCN-SEQ ID NO: 52), gamma-secretase subunit APH-1A (AAVFFGCTFVAFGPAFALFLI-SEQ ID NO: 53; VIILVAGAFFWLVSLLLASVV-SEQ ID NO: 54; YGLLIFGAAVSVLLQEVFRFA-SEQ ID NO: 55; YVSGLSFGIISGVFSVINILA-SEQ ID NO: 56; TSAFLTAAIILLHTFWGVVFF-SEQ ID NO: 57; ATMILVPLLGIQFVVFPW-SEQ ID NO: 58; LLPIYAVTVSMGLWAFITAGG-SEQ ID NO: 59), or adipnectin receptor protein 2 (NIWTHLLGCVFFLCLGIFYMF-SEQ ID NO: 60; VVFGLFFLGAILCLSFSWLFH-SEQ ID NO: 61; LFSKLDYSGIALLIMGSFVPW-SEQ ID NO: 62; CFIYLIVICVLGIAAIIVSQW-SEQ ID NO: 63; YRGVRAGVFLGLGLSGIIPTL-SEQ ID NO: 64; QIGWLMLMASLYITGAALYAA-SEQ ID NO: 65; QLFHIFVVAGAFVHFHGVSNL-SEQ ID NO: 66); human oncostatin M (MGVLLTQRTLLSLVLALLFPSMASM), VSV-G (MKCLLYLAFLFIGVNC), Osteonectin (MRAWIFFLLCLAGRALA), secrecon (MWWRLWWLLLLLLLLWPMVWA), human Ig kappa (MDMRVPAQLLGLLLLWLRGARC), human Ig heavy (MDILCSTLLLLTVPSGVLS; MKHLWFLLLWCQLPDVGVL; MDWTXXXXFLVAAATRVHS; MDWTWRILFLVAAATGAHS; MDWTWRVFCLLAVAPGAHS; MEFGLSWLFLVAILKGVQC), tPA (MDAMKRGLCCVLLLCGAVFVSPS), human chymotrypsinogen (MAFLWLLSCWALLGTTFG), human trysinogen-2 (MNLLLILTFVAAAVA), human IL-2 (MYRMQLLSCIALSLALVTNS), guassia luciferase (MGVKVLFALICIAVAEA), human serum albumin (MKWVTFISLLFSSAYS), influenza haemagglutinin (MKTIIALSYIFCLVLG), human insulin (MALWMRLLPLLALLALWGPDPAAA), silkworm fibroin LC (MKPIFLVLLVVTSAYA), CD33 (MPLLLLLPLLWAGALA).

### The Cancer Targeting Domain

In one embodiment, the cancer targeting domain identifies and binds to an antigen on cancer cells once the immune cells are modified into chimeric antigen receptor (CAR) immune cells by the recombinant CAR fusion protein.

The cancer targeting domain may be a cancer capturing domain, a cancer binding domain, an anti-cancer receptor domain, a cancer receptor binding domain, or a cancer receptor targeting domain.

The cancer targeting domain can be an antibody, an antibody fragment or an antigen-binding fragment. In one embodiment, the cancer targeting domain may be PD-L1 antigen binding domain, anti-PD-L1 Vh (EVOLVESGGGLVQPGGSLRLSCAASGFTFSDSWIHWVRQAPGKGLEWVAWISPYG GSTYYADSVKGRFTISADTSKNTAYLQMNSLRAEDTAVYYCARRHWPGGFDYWGQ GTLVTVSSASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKKVEPKSCDKT HTCPPCPAPELLGGPSVFLFPPKPKDTLMISRTPEVTCVVVDVSHEDPEVKFNWYVD GVEVHNAKTKPREEQYASTYRVVSVLTVLHQDWLNGKEYKCKVSNKALPAPIEKTI SKAKGQPREPQVYTLPPSREEMTKNQVSLTCLVKGFYPSDIAVEWESNGQPENNYKT TPPVLDSDGSFFLYSKLTVDKSRWQQ GNVFSCSVMHEALHNHYTQKSLSLSPGK; SEQ ID NO: 67), a HER2, CD19, CD33, CD20, CD22, CD30, CD33, CD138, CD123, CD70, CD79b, CD37, FOLR1, TROP2, DLL3, ENPP3, CA6, B cell maturation antigen (BCMA), carbonic anhydrase IX (CAIX), CD171, carcinoembryonic antigen (CEA), ERBB2, EGFR, EGFRVIII, GD2, αFR, GP100, Lewis Y, melanoma antigen recognized by T cells 1 (MART 1), melanoma antigen A3 (MAGEA3), NYSEO1, P53, prostate specific membrane antigen (PSMA), mucin 16 (MUC16), glypican 3 (GPC3), Nectin 4, mesothelin antigen binding domain.

### The Hinge Region

In one embodiment, the recombinant CAR fusion protein further comprises a hinge region. In some aspects, the hinge region can be located between the cancer targeting domain and the transmembrane domain. The hinge region may increase the distance of the cancer targeting domain and the transmembrane domain, and provide flexibility. In one embodiment, the hinge region may contain 12-45 amino acids. For instance, the hinge region may be CD8 hinge (FVPVFLPAKPTTTPAPRPPTPAPTIASQPLSLRPEACRPAAGGAVHTRGLDFACD; SEQ ID NO: 68), CD28, IgG1, and IgG4.

### The Transmembrane Domain

In one embodiment, during the process of exocytosis of the rest of the recombinant CAR fusion protein domains toward the outer cell membrane (plasma membrane), the transmembrane domain is positioned in the cell membrane, and thus the intracellular signaling domain is positioned beneath the inner cell membrane.

The transmembrane domain may be a membrane anchoring domain, or a membrane localization domain.

In some aspects, the transmembrane domain can be CD28, CD3, CD4, CD7, CD8, FcεR1γ, ICOS, H2-Kb, NKG2D, CD16, NKp44, or NKp46.

### The Intracellular Signaling Domain

In one embodiment, the intracellular signaling domain comprises a CD3 zeta (CD3ζ) signaling domain, and a co-stimulatory signaling region selected from the group consisting of 2B4, DAP12, GITR, CD137, OX40, CD27, ICOS, CD40, 41BB, and DAP10.

### The Tag Sequence

In one embodiment, the recombinant CAR fusion protein further comprises a tag sequence. In some aspects, the tag sequence can be located at the C-terminal or N-terminal end of the recombinant CAR fusion protein, optionally via a linker. The linker may be a serine-glycine linker such as GGGGS (SEQ ID NO: 69), GGGSS (SEQ ID NO: 70), GGGSG (SEQ ID NO: 71), or multiple variants thereof such as GGGGSGGGGS (SEQ ID NO: 72) or (GGGGS-SEQ ID NO: 69) m, (GGGSS-SEQ ID NO: 70) m, (GGGSG-SEQ ID NO: 71) m, where m is an integer from 1 to 5, from 1 to 4 or from 1 to 3. In a preferred embodiment m is 2.

In some aspects, the tag sequence can be, but is not limited to, a histidine tag, glutathione-S-transferase tag, maltose binding protein tag, Strep tag, or hemagglutinin tag. Examples of these tags can be, but are not limited to histidine tag (HHHHHH-SEQ ID NO: 73; HHHHHHHH-SEQ ID NO: 74; HHHHHHHHHH-SEQ ID NO: 75), glutathione-S-transferase tag (MSPILGYWKI KGLVQPTRLLLEYLEEKYEEHLYERDEGDK WRNKKFELGLEFPNLPYYIDGDVKLTQSMAIIRYIADKHNMLGGCPKERAEISMLEG AVLDIRYGVSRIA YSKDFETLKVDFLSKLPEMLKMFEDRLCHKTYLNGDHVTHPDF MLYDALD VVLYMDPMCL DAFPKLVCFKKRIEAIPQID KYLKSSKYIAWPLQGWQATF GGGDHPPK; SEQ ID NO: 76), maltose binding protein tag (MKIKTGARILALSALTTMMFSASALAKIEEGKLVIWINGDKGYNGLAEVGKKFEKD TGIKVTVEHPDKLEEKFPQVAATGDGPDIIFWAHDRFGGYAQSGLLAEITPDKAFQD KLYPFTWDAVRYNGKLIAYPIAVEALSLIYNKDLLPNPPKTWEEIPALDKELKAKGK SALMFNLQEPYFTWPLIAADGGYAFKYENGKYDIKDVGVDNAGAKAGLTFLVDLIK NKHMNADTDYSIAEAAFNKGETAMTINGPWAWSNIDTSKVNYGVTVLPTFKGQPSK PFVGVLSAGINAASPNKELAKEFLENYLLTDEGLEAVNKDKPLGAVALKSYEEELAK DPRIAATMENAQKGEIMPNIPQMSAFWYAVRTAVINAASGRQTVDEALKDAQTRIT K; SEQ ID NO: 77), or Strep tag (WSHPQFEK; SEQ ID NO: 78).

The tag sequence can be utilized to isolate recombinant CAR protein from E. Coli or other cells during the protein production and to verify presence of recombinant CAR protein in immune cells.

### The Linker

In one embodiment, the linker may be also included between the immune cell targeting domain and the cleavable peptide. For instance, the linker may be a serine-glycine linker such as GGGGSGGGGSGGGGS (SEQ ID NO: 79), GGGGS (SEQ ID NO: 69), GGGSS (SEQ ID NO: 70), GGGSG (SEQ ID NO: 71), or multiple variants thereof such as GGGGSGGGGS (SEQ ID NO: 72) or (GGGGS-(SEQ ID NO: 69)) m, (GGGSS-(SEQ ID NO: 70)) m, (GGGSG-(SEQ ID NO: 71)) m, where m is an integer from 1 to 5, from 1 to 4 or from 1 to 3. In a preferred embodiment m is 2.

In one embodiment, the recombinant CAR fusion protein comprises the sequence of SEQ ID NO: 1.

In one embodiment, the recombinant CAR fusion protein comprises the sequence of SEQ ID NO: 80.

2. The Method of Modifying an Immune Cell into a Chimeric Antigen Receptor (CAR) Immune Cell

The present disclosure also provides a method of modifying an immune cell into a chimeric antigen receptor (CAR) immune cell, comprising treating the immune cell with the recombinant CAR fusion protein discussed above.

In one embodiment, the above method may be applied in both in vivo, and ex vivo.

In the in vivo application, the immune cell targeting domain may be an antibody, antibody fragment or an antigen-binding fragment targeting a specific immune cell which is to be modified into a CAR immune cell. For instance, in order to modify NK cells into CAR NK cells in the in vivo application, the immune cell targeting domain may be a NK cell targeting domain. In addition, in order to modify T cells into CAR T cells in the in vivo application, the immune cell targeting domain may be a T cell targeting domain.

The NK cell targeting domain may be an antibody, antibody fragment or an antigen-binding fragment targeting NK cells. For instance, the NK cell targeting domain may be NK cell-targeting antibody, NK cell-targeting-scFv antibody, NK cell-targeting bispecific scFv antibody, NK cell-targeting nanobody, NK cell-targeting camel antibody, or a peptide comprising a NK cell-targeting antibody-V_{H}, V_{L}, V_{HH}, hinge, CH1, CH2, CH3, or CL. The NK cell targeting antibody can be anti-NKp30, anti-NKp44, anti-NKp46, anti-NKp80, anti-NKG2A, anti-NKG2C, anti-NKG2D, anti-CD16, anti-CD56, anti-KIR-s, anti-CD122 anti-PD-1, anti-TIGIT, anti-LAG3, or anti-KLRG.

In addition, the T cell targeting domain may be a T cell-targeting antibody, T cell-targeting-scFv antibody, T cell-targeting bispecific scFv antibody, T cell-targeting nanobody, T cell-targeting camel antibody, a peptide comprising a T cell-targeting antibody-V_{H}, hinge, CH1, CH2, CH3, or CL. The T cell targeting antibody can be anti-CD3, anti-CD8, anti-CD4, anti-CD45R, anti-CD40, anti-CD25, anti-OX-40, anti-NKG2D, anti-NKp46, anti-NKp30, anti-PD-1, anti-TIM3, anti-TIGIT, anti-LAG3, or anti-CTLA4.

In the ex vivo application, since specific immune cells (which are to be modified into CAR immune cells) may be directly treated with the recombinant CAR fusion protein, the immune cell targeting domain may be also selected from any protein-transduction domain, which is not specific to the target immune cell, but allows non-specific transduction of the recombinant CAR fusion protein, into any cells. In one embodiment, the immune cell targeting domain for ex vivo application (which is not specific to certain immune cells) may include Tat or a Tat peptide, poly-arginine, antennapedia (Antp) or Antp peptide, penetratin, SAP, PTD-5, K-FGF (SN50 peptide), HIV-1 Rev, FHV, HTLV-II, NLS, transportan, pVEC. Examples of these domains can be, but are not limited to Tat (YGRKKRRQRRR; SEQ ID NO: 2) or a Tat peptide (RKKRRQRRR; SEQ ID NO: 3), poly-arginine (RRRRRR; SEQ ID NO: 4; RRRRRRRR; SEQ ID NO: 5; RRRRRRRRR; SEQ ID NO: 6), antennapedia (Antp) or Antp peptide (RQIKIWFQNRRMKW; SEQ ID NO: 7), penetratin (RQIKIWFQNRRMKWKK; SEQ ID NO: 8), SAP (VRLPPPVRLPPPVRLPPP; SEQ ID NO: 9), PTD-5 (RRQRRTSKLMKR; SEQ ID NO: 10), K-FGF (SN50 peptide) (AAVALLPAVLLALLAP; SEQ ID NO: 11), HIV-1 Rev (TRQARRNRRRRWRERQR; SEQ ID NO: 12), FHV (RRRRNRTRRNRRRVR; SEQ ID NO: 13), HTLV-II (TRRQRTRRATTNR; SEQ ID NO: 14), NLS (KRPAAIKKAGQAKKKK; SEQ ID NO: 15), transportan (GWTLNSAGYLLGKINLKALAALAKKIL; SEQ ID NO: 16), pVEC (LLIILRRRIRKQAHAHSK; SEQ ID NO: 17). See also Table 1 above.

In one embodiment, the recombinant CAR fusion protein may be treated to immune cells at a concentration which is efficient to modify the immune cells into CAR immune cells in view of the uptake efficiency of the immune cells. For instance, the lower limit of such a concentration may be a concentration which is sufficient for the modified CAR immune cells to show a therapeutic effect for cancer. In addition, the upper limit of such a concentration may be a concentration where the uptake of the recombinant CAR fusion protein by immune cells are saturated. For instance, the recombinant Car fusion protein may be in a concentration of 1,000 nM to 2,000 nM. In another embodiment, the recombinant Car fusion protein may be in a concentration of about 1,000 nM to about 2,000 nM. In another embodiment, the lower limit may be about 1,100 nM, about 1,200 nM, about 1,300 nM, about 1,400 nM, or about 1,500 nM. In another embodiment, the upper limit may be about 1,900 nM, about 1,800 nM, about 1,700 nM, or about 1,600 nM. In another embodiment, the recombinant Car fusion protein may be in a concentration of about 1,000 nM.

### 3. The Method of Treating Cancer by Administering the CAR Immune Cell to a Subject in Need Thereof

The present disclosure also provides a method of treating cancer comprising administering a CAR immune cell, which was prepared by treating an immune cell with the recombinant CAR fusion protein of the present disclosure, to a subject in need thereof.

In one embodiment, the CAR immune cell is prepared by the ex vivo application discussed in the present disclosure, and is administered to the subject for treating cancer.

In one embodiment, the cancer being treated can be kidney cancer, spleen cancer, lung cancer, liver cancer, breast cancer, lung cancer, B cell cancer, prostate cancer, lymphoma, Chronic lymphocytic leukemia (CLL), B cell acute lymphocytic leukemia (B-ALL), Non-Hodgkin's lymphoma (NHL), Acute lymphoblastic leukemia (ALL), myeloid malignancies, multiple myeloma, renal cell carcinoma (RCC), EGFR-positive solid tumors, Glioblastoma, neuroblastoma, Ewing's sarcoma, Osteosarcoma, acute myeloid leukemia (AML), melanoma, oesophageal, Synovial sarcoma, sarcoma, colorectal cancer, hepatocellular carcinoma, non-small cell lung cancer, pancreatic carcinoma, triple-negative invasive breast cancer, ovarian cancer, or mesothelioma.

In another embodiment, the CAR immune cell is administered to the subject in an amount which is therapeutically effective to treat cancer.

In another embodiment, the above method may further include administering a therapeutic agent. For instance, the therapeutic agent may be chemotherapy, proteasome inhibitors, immunomodulatory agents, histone deacetylase inhibitors, monoclonal antibodies, bispecific antibodies, recombinant antibodies, or immune checkpoint inhibitors.

The CAR immune cell can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, such as a blood cancer or a solid tumor.

The effective amount can be administered in one or more administrations, applications or dosages and is not intended to be limited to a particular formulation or administration route. In an embodiment, administering is via a course of treatment comprising a plurality of treatment cycles and a plurality of rest periods.

### 4. The Method of Treating Cancer by Administering the Recombinant CAR Fusion Protein to a Subject in Need Thereof

The present disclosure also provides a method of treating cancer comprising administering the recombinant CAR fusion protein to a subject in need thereof.

In one embodiment, the cancer being treated can be kidney cancer, spleen cancer, lung cancer, liver cancer, breast cancer, lung cancer, B cell cancer, prostate cancer, lymphoma, Chronic lymphocytic leukemia (CLL), B cell acute lymphocytic leukemia (B-ALL), Non-Hodgkin's lymphoma (NHL), Acute lymphoblastic leukemia (ALL), myeloid malignancies, multiple myeloma, renal cell carcinoma (RCC), EGFR-positive solid tumors, Glioblastoma, neuroblastoma, Ewing's sarcoma, Osteosarcoma, acute myeloid leukemia (AML), melanoma, oesophageal, Synovial sarcoma, sarcoma, colorectal cancer, hepatocellular carcinoma, non-small cell lung cancer, pancreatic carcinoma, triple-negative invasive breast cancer, ovarian cancer, or mesothelioma.

In another embodiment, the recombinant CAR fusion protein may be administered to the subject at a concentration which is efficient to modify the immune cells into CAR immune cells in view of the uptake efficiency of the immune cells. For instance, the lower limit of such a concentration may be a concentration which is sufficient for the modified CAR immune cells to show a therapeutic effect for cancer. In addition, the upper limit of such a concentration may be a concentration where the uptake of the recombinant CAR fusion protein by immune cells are saturated. For instance, the recombinant Car fusion protein may be in a concentration of 100 nM to 2,000 nM. In another embodiment, the recombinant Car fusion protein may be in a concentration of about 100 nM to about 2,000 nM. In another embodiment, the lower limit may be about 1,100 nM, about 1,200 nM, about 1,300 nM, about 1,400 nM, or about 1,500 nM. In another embodiment, the upper limit may be about 1,900 nM, about 1,800 nM, about 1,700 nM, or about 1,600 nM. In another embodiment, the lower limit may be about 110 nM, about 120 nM, about 130 nM, about 140 nM, or about 150 nM. In another embodiment, the upper limit may be about 500 nM, about 490 nM, about 480 nM, or about 470 nM. In one embodiment, the recombinant Car fusion protein may be in a concentration of about 100 nM to 500 nM. In another embodiment, the recombinant Car fusion protein may be in a concentration of about 1,000 nM.

The recombinant CAR fusion protein can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a cancer, such as a blood cancer or a solid tumor.

The effective amount can be administered in one or more administrations, applications or dosages and is not intended to be limited to a particular formulation or administration route. In an embodiment, administering is via a course of treatment comprising a plurality of treatment cycles and a plurality of rest periods.

### 5. The Method of Treating Cancer by Administering the Recombinant CAR Fusion Protein and CAR Immune Cell to a Subject in Need Thereof

The present disclosure also provides a method of treating cancer by administering both of the recombinant CAR fusion protein and CAR immune cell discussed above. In particular, in one embodiment, the present disclosure provides a method of treating cancer comprising administering to a subject in need thereof: (i) a recombinant chimeric antigen receptor (CAR) fusion protein comprising an immune cell targeting domain, a cleavable peptide, a membrane targeting domain, a cancer targeting domain, a transmembrane domain, and an intracellular signaling domain; and (ii) a CAR immune cell.

In one embodiment, the CAR immune cell is prepared by treating an immune cell with the recombinant CAR fusion protein. In another embodiment, the CAR immune cell may be CAR immune cells prepared by any other methods.

### EXAMPLES

### Example 1

### (A) Preparation of the Recombinant CAR Fusion Protein Targeting PD-L1

A recombinant CAR fusion protein targeting PD-L1 was prepared in the following structure.
anti-NKp46 Vh-IgG Hinge-IgG CH2-IgG CH3-linker-Furin cleavage site-membrane targeting-anti-PD-L1 Vh-CD8 Hinge-NKG2D TM-2B4-CD3 zeta-His tag

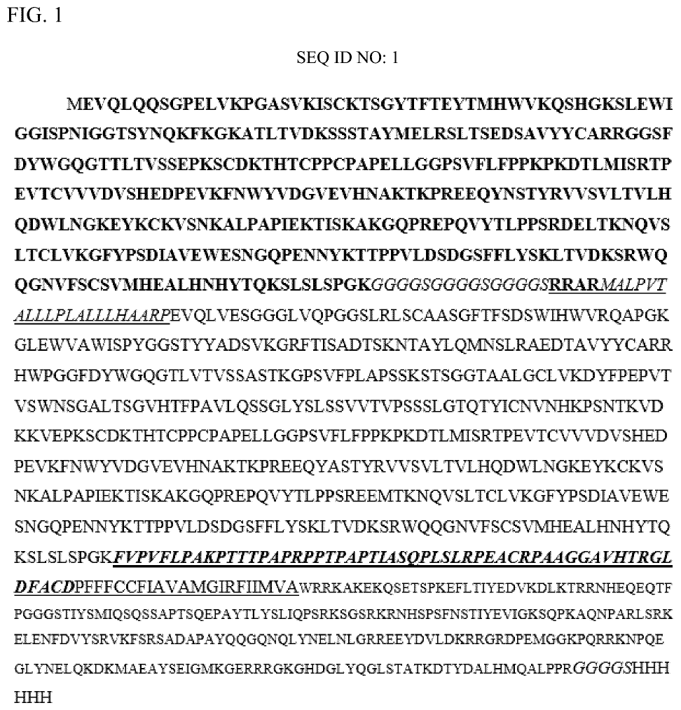

In particular, the amino acid sequence of the recombinant CAR fusion protein targeting PD-L1 was as shown in FIG. 1 (SEQ ID NO: 1). The bold letters next to M represent anti-NKp46 Vh-IgG Hinge-IgG CH2-IgG CH3 (i.e., the immune cell targeting domain). The italicized letters represent a GGGGSGGGGSGGGGS (SEQ ID NO: 79) linker and a GGGGS (SEQ ID NO: 69) linker. The bold underlined letters represent a Furin cleavage site (RRAR (SEQ ID NO: 34); i.e., the cleavable peptide). The italicized underlined letters represent a membrane targeting domain. The regular capitalized letters represent anti-PD-L1 Vh (i.e., the cancer targeting domain). The bold italicized letters represent CD8 hinge (i.e., the hinge region). The underlined letters represent NKG2D (i.e., the transmembrane domain). The smaller letters represent 2B4-CD3 zeta (i.e., the intracellular signaling domain). HHHHHH (SEQ ID NO: 73) at the C-terminal end is the histidine tag (i.e., the tag sequence).

### (B) SDS-PAGE and Western Blot Analysis

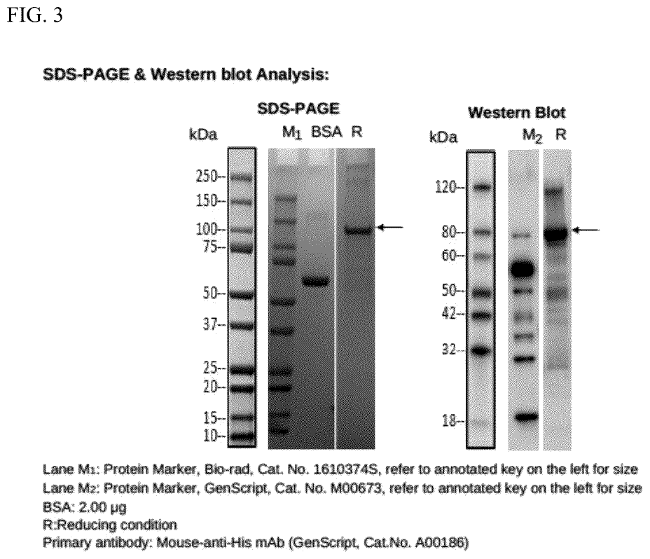

The recombinant CAR fusion protein was run on SDS-PAGE and western blot to visualize its molecular weight. The molecular weight of the recombinant CAR fusion protein is 127 kDa, and SDS-PAGE and western blot data show that the band of recombinant CAR fusion protein is around 120 kDa molecular weight marker, indicating well-production of the recombinant CAR fusion protein. See FIG. 3.

### (C) Confirmation of the Uptake of the Recombinant CAR Fusion Protein by NK Cells

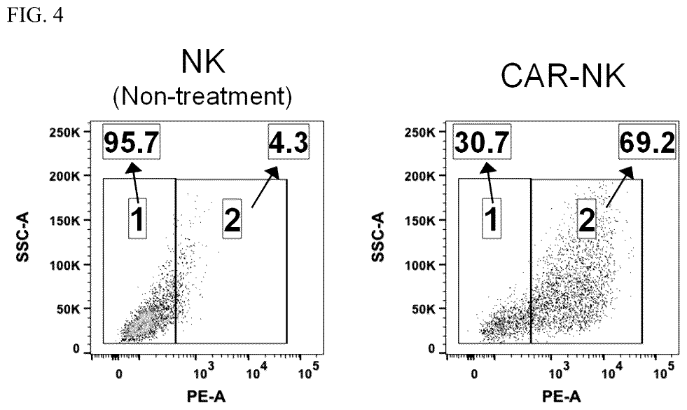

The prepared recombinant CAR fusion protein was incubated with NK-92 cells (1 μM of the CAR fusion protein, 1×10⁶ cells/0.5 mL) for 24-hour. The cells were collected and fixed to be permeabilized. Then, PE-conjugated anti-his tag antibody was treated to label his-tag on the recombinant CAR fusion protein. As shown in FIG. 4, it showed that the NK cells treated with the recombinant CAR fusion protein showed higher PE intensity due to its his-tag.

### (D) The Recombinant CAR Fusion Protein Transduction Optimization

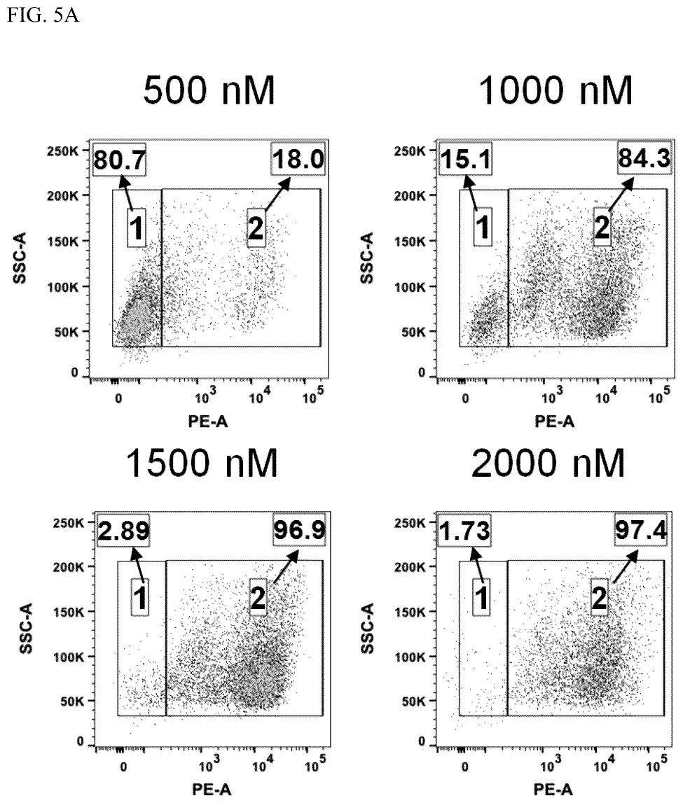

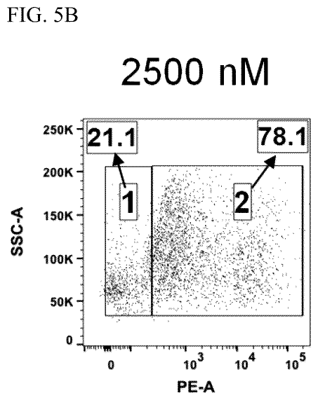

To optimize concentration for the recombinant CAR fusion protein transduction, the recombinant CAR fusion protein with various concentration was treated to NK-92 cells (1×10⁶ cells/0.5 mL) for 24-hour. Protein transduction was confirmed with same method as discussed under Example 1 (B) above. As shown in FIGS. 5A and 5B, it was confirmed that protein uptake was saturated over 1500 nM. It was speculated that the difference of uptake efficiency between 1000 nM and 1500 nM was not significant to bring the difference on therapeutic efficacy.

### (E) Target (PD-L1) Binding Ability of the Recombinant CAR Fusion Protein

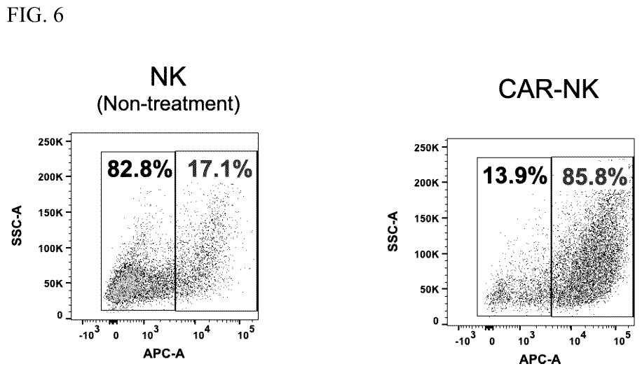

After the CAR-NK cells were generated, PD-L1 protein (20 ng/mL) was treated to both of the CAR-NK cells and the NK cells and incubated for 2-hour. Because PD-L1 protein is biotinylated, the PD-L1 protein-bound cells can be identified using APC-streptavidin. The cells were not permeabilized at this experiment in order to stain exterior biotin-tag which was conjugated on PD-L1 protein. As shown in FIG. 6, it showed that the CAR-NK cells had higher APC intensity, which indicated that more PD-L1 protein was bound to the CAR-NK cells due to the recombinant CAR fusion protein.

(F) Cytokine Release from NK Cells or CAR-NK Cells after PD-L1 Binding Through PD-L1 Protein Treatment.

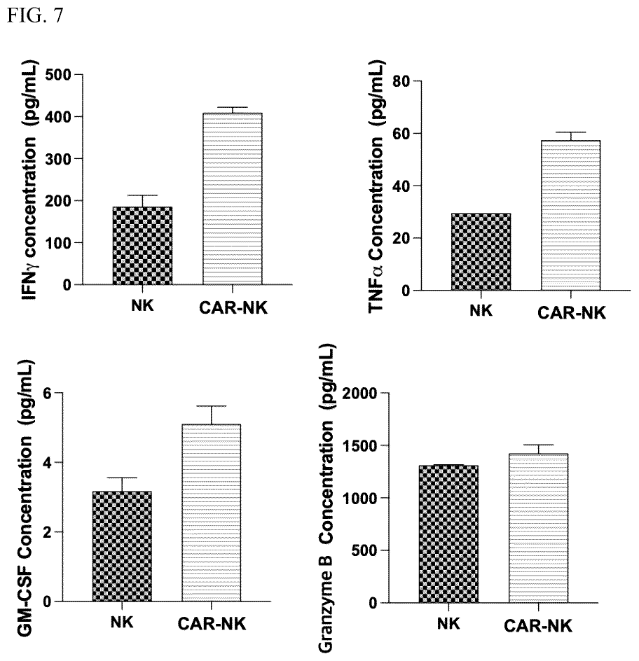

In order to verify the increased anti-cancer activity owing to the recombinant CAR fusion protein, cytotoxic/cytolytic cytokine secretion upon PD-L1 binding was analyzed. After PD-L1 binding to the NK cells or CAR-NK cells (2-hour treatment of 20 ng/ml PD-L1 protein), cell supernatant was harvested and ran ELISA. As shown in FIG. 7, it was confirmed that cytokine secretion of the CAR-NK cells was overly increased as compared to that of the NK cells. This result implied that the CAR-NK cells would have stronger anti-cancer activity over the NK cells.

### (G) Anti-Cancer Activity of CAR-NK Cell: Cancer Cell Death

Anti-cancer activity of the CAR-NK cells and NK cells were compared through induction of cancer cell death. In particular, cancer cells treated with the NK cells, cancer cells treated with the CAR-NK cells, and cancer cells which were not treated with the NK cells or CAR-NK cells were compared. The experiment condition was as follows.
CellTracker Blue™ labelled MDA-MB-231 cancer cells
Effector:Target (E:T)=5:1
4-hour co-incubation of CellTracker Blue labelled-MDA-MB-231 & NK/CAR-NK

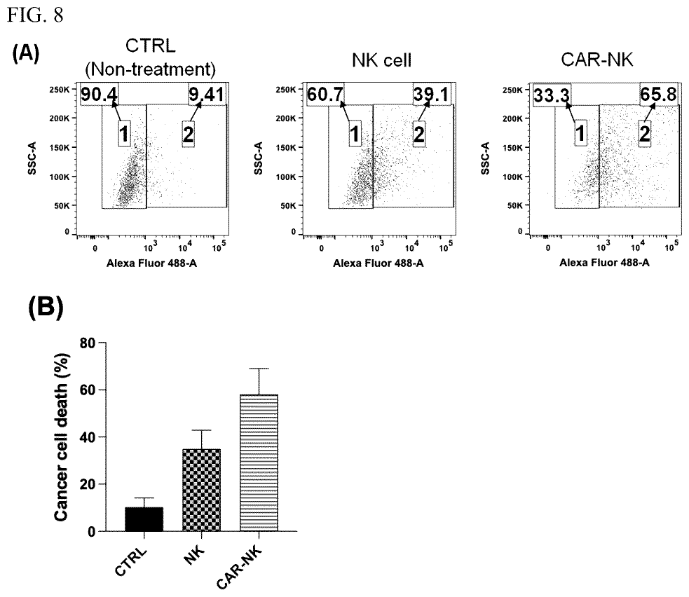

After co-incubation, cells were harvested and stained Alexa Fluor-488 Annexin V to label dead cells. The cells were run through flow cytometry and the population of CellTracker Blue labelled cells (cancer cells) were analyzed intensity of Alexa Fluor 488 (A of FIG. 8). Alexa Fluor 488-Annexin V stained cells indicated dead cancer cells. The more population in number 2 box, the more dead cancer cells. Populations of number 2 box in A of FIG. 8 were summarized in B of FIG. 8. These results referred that the CAR-NK cells had stronger anti-cancer activity as compared to the NK cells.

### (H) Anti-Cancer Activity of CAR-NK Cell: NK Cells' Cytokines

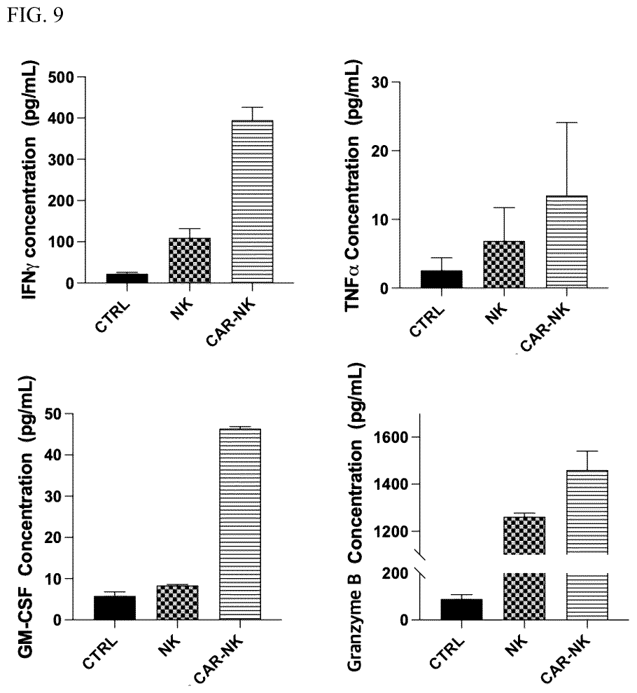

Cytokine secretion from the NK cells and the CAR-NK cells were measured from the cell supernatant after 4 hours co-incubation of cancer cells and the NK cells and the CAR-NK cells. Here, the control group denotes cancer cells which were not treated with the NK cells or CAR-NK cells. The results are shown in FIG. 9. Cytotoxic/cytolytic cytokine were more secreted from and the CAR-NK cells upon cancer cell engraftment than the NK cells. It was confirmed that the increased cancer cell death of the CAR-NK cells was induced by the increased cytokine secretion. Consequently, it was confirmed that the recombinant CAR fusion protein of the present disclosure can modify NK cells to have a CAR system without viral vector-mediated gene transfection and genetic expression time, and consequently, bring the stronger anti-cancer activity.

### (I) In Vitro Retention of CAR-NK Generated by the Recombinant CAR Fusion Protein

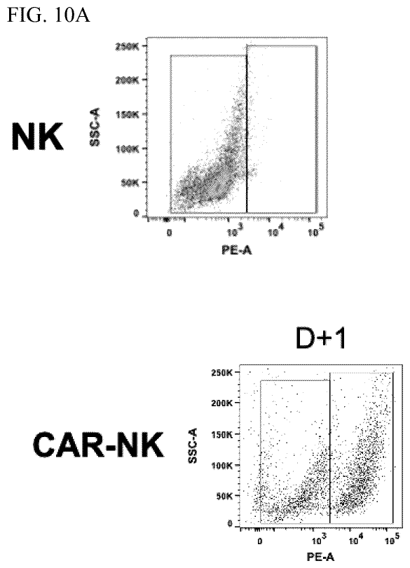

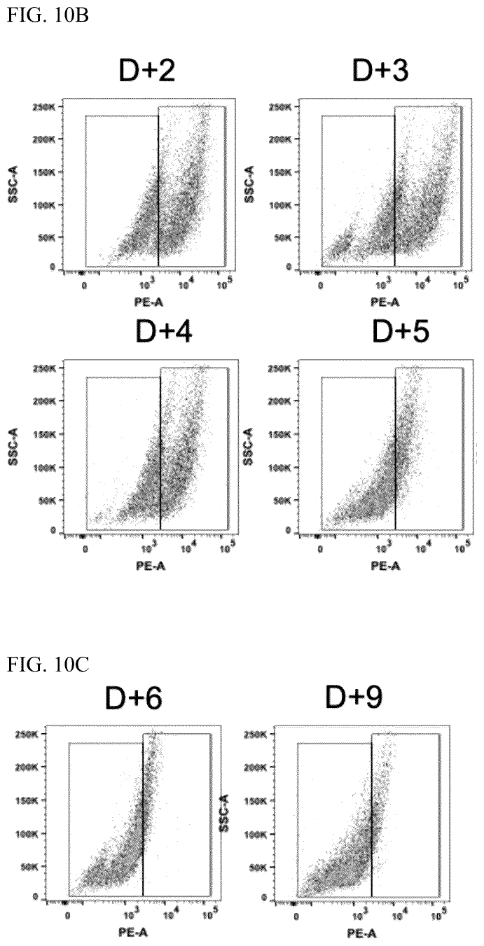

The retention of CAR-NK was confirmed by the intracellular presence of his-tag at the various time points. CAR-NK cells were generated under the same conditions as other in vitro experiments. The unbound recombinant CAR fusion protein was removed on D+1 using centrifuge process. After the removal of recombinant CAR fusion protein, CAR-NK cells were incubated in the basic culture condition for NK cells. CAR-NK cells were collected at the various time points after the treatment of recombinant CAR fusion protein. The collected cells were fixed and permeabilized to label his-tag with PE-conjugated anti-his tag antibody. CAR-NK cells were detected until D+9. See FIGS. 10A, 10B and 10C.

The transduction conditions of the recombinant CAR fusion protein were 1 μM recombinant CAR fusion protein, 1×10⁶ cells/0.5 mL, NK-92 culture medium.

### (J) In Vivo Tumor Inhibition by the Recombinant CAR Fusion Protein

5 million MDA-MB-231 cells were subcutaneously inoculated in humanized NSG mice. Control group received DPBS, and test groups received 1.7 mg/Kg or 11 mg/Kg recombinant CAR fusion protein intravenously injected once a week for 4 weeks. Tumor volume and body weight was measured twice a week. Tumor volume was measured by caliper measurements (v=0.5ab2; a=long length, b=short length). (A) Relative tumor volume, (B) Relative body weight, and (C) Survival rate. Circle: control group, Square: 1.7 mg/Kg recombinant CAR fusion protein, and Triangle: 11 mg/Kg recombinant CAR fusion protein. (D) Individual relative tumor volume for each group.

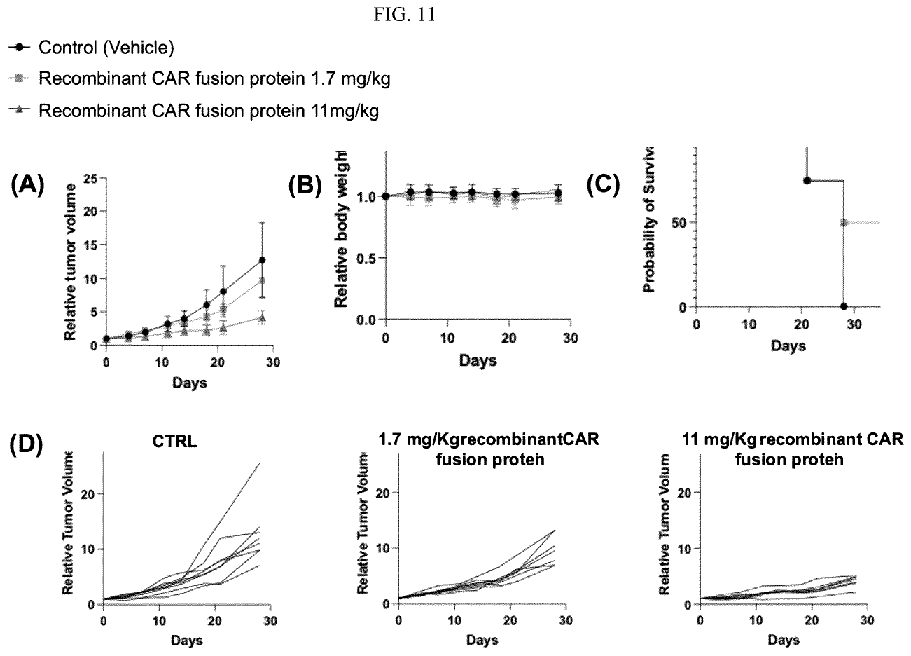

The group of 11 mg/Kg recombinant CAR fusion protein showed significant tumor inhibition and improved survival rate. The recombinant CAR fusion protein did not affect body weight, indicating safety and non-toxicity. These in vivo experiments demonstrate the therapeutic efficacy and dose-dependency of the in vivo CAR protein. See FIG. 11.

### (K) Human Cytokine in Tumor Tissues and Plasma

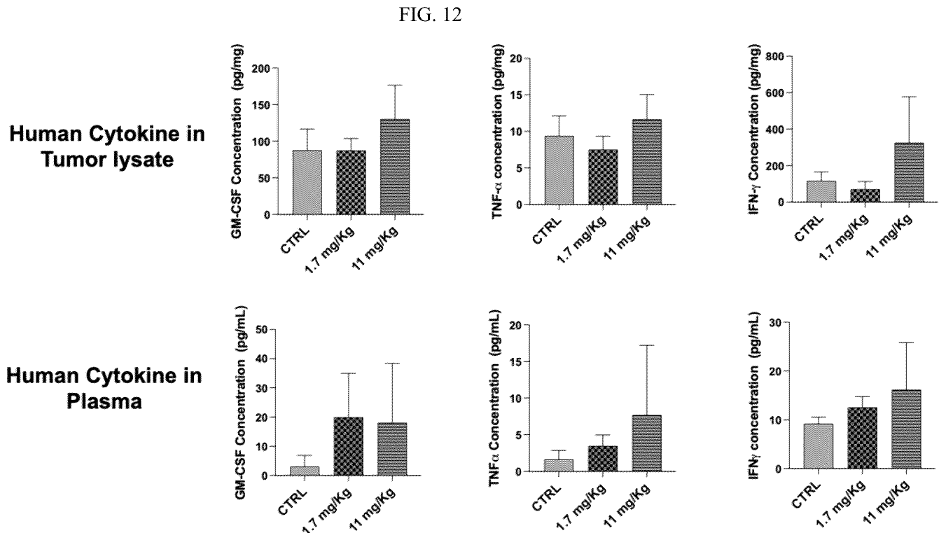

After the measurement of tumor volume and body weight on D+28, animals were sacrificed to collect blood and organs (tumor, liver, kidney, spleen, and lung). Plasma was subsequently harvested by centrifuge with a gradient medium. To obtain tumor lysate, tumor tissues were homogenized and lysed in RIPA buffer. The supernatant was collected by centrifugation to remove cell debris. Tumor lysate and plasma were run on ELISA kits for human cytokines. Recombinant CAR fusion protein-treated groups demonstrated increasing cytokine secretion. It was hypothesized that the increased concentration of cytokines inhibits tumor progression. This result implies that the recombinant CAR fusion protein induces the activation of the immune system and accounts for its therapeutic efficacy. See FIG. 12.

### (L) Recombinant CAR Fusion Protein-Modification on Blood Human NK and Human T Cells

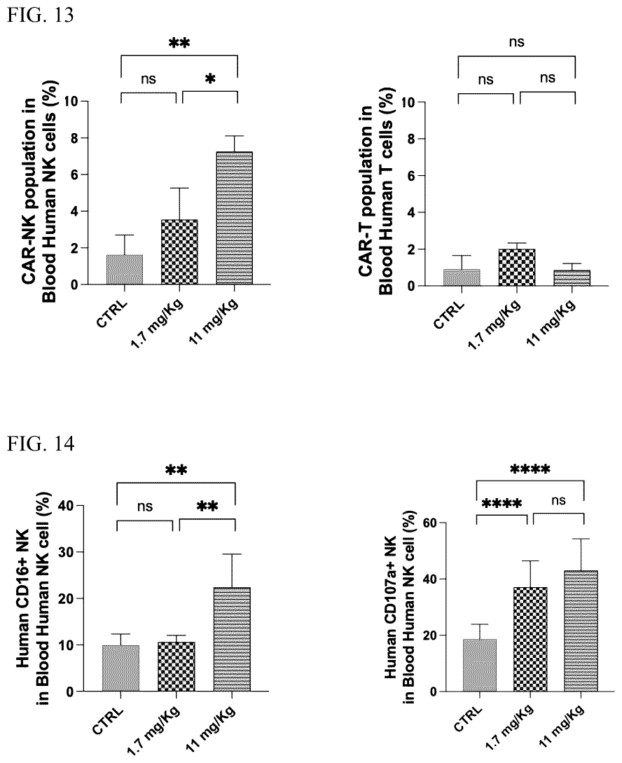

Blood was collected on D+28 which was 7 days after the last injection of the recombinant CAR fusion protein. Immune cells in the blood were harvested through centrifugation with gradient medium, human NK cells or human T cells were isolated. The isolated human NK and human T cells were fixed and permeabilized to detect in vivo CAR-NK cells or in vivo CAR-T cells, generated by the recombinant CAR fusion protein, by labeling his-tag on the recombinant CAR fusion protein with PE anti-his tag antibody. The in vivo CAR-NK cells were significantly detected in the recombinant CAR fusion protein-injected groups, and there was dose-dependency. However, the difference in the in vivo CAR-T population was negligible between the groups. This result indicates that the current recombinant CAR fusion protein specifically modifies NK cells because of its NK cell-targeting domain. Since the recombinant CAR fusion protein has a modular design, the NK cell-targeting domain can be replaced with a T cell-targeting domain or domain that simultaneously targets both NK cell and T cell for in vivo CAR-T application or simultaneous application of in vivo CAR-NK and CAR-T. See FIG. 13.

### (M) NK Cells Become More Cytolytic Upon the Injection of Recombinant CAR Protein

Human NK cells isolated from blood were analyzed for their cytolytic characteristics. CD16+ NK cells and CD107a+ NK cells have cytotoxic/cytolytic features, which are antibody-dependent cellular cytotoxicity (ADCC) and degranulation, respectively. The isolated human NK cells were treated with anti-human CD16 antibody or anti-human CD107a antibody. The population of human CD16+ NK cells was increased in the 11 mg/Kg recombinant CAR fusion protein-injected group. The population of human CD107a+ NK cells was increased in both of the recombinant CAR fusion protein-injected groups. The results suggest that the recombinant CAR fusion protein stimulates NK cells to have cytolytic characteristics. See FIG. 14.

### (N) Initial Liver Toxicity Test

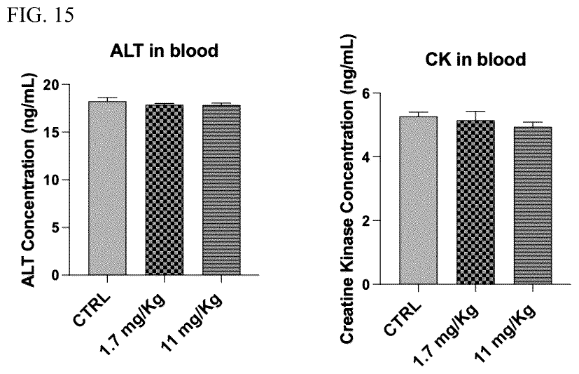

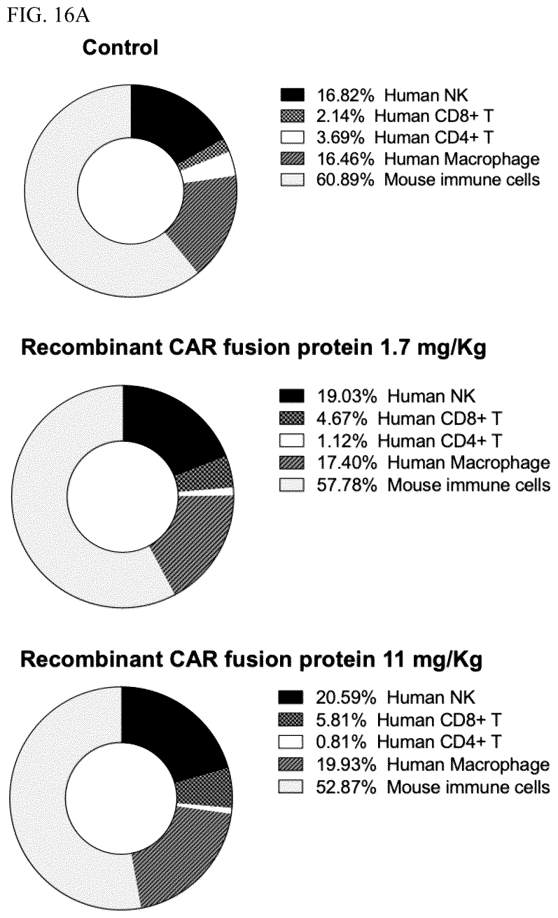

The levels of alanine transaminase (ALT) and creatine kinase (CK) in blood were analyzed to determine whether the recombinant CAR fusion protein causes liver toxicity. Plasma was run on ELISAs to measure the concentrations of ALK and CK. The injection of the recombinant CAR fusion protein did not result in the elevation of the level of ALT or CK. This result indicates that the injection of the recombinant CAR fusion protein does not cause the liver toxicity. See FIG. 15.

### (O) Recombinant CAR Fusion Protein Generates a Comprehensive Immune Response in the TME

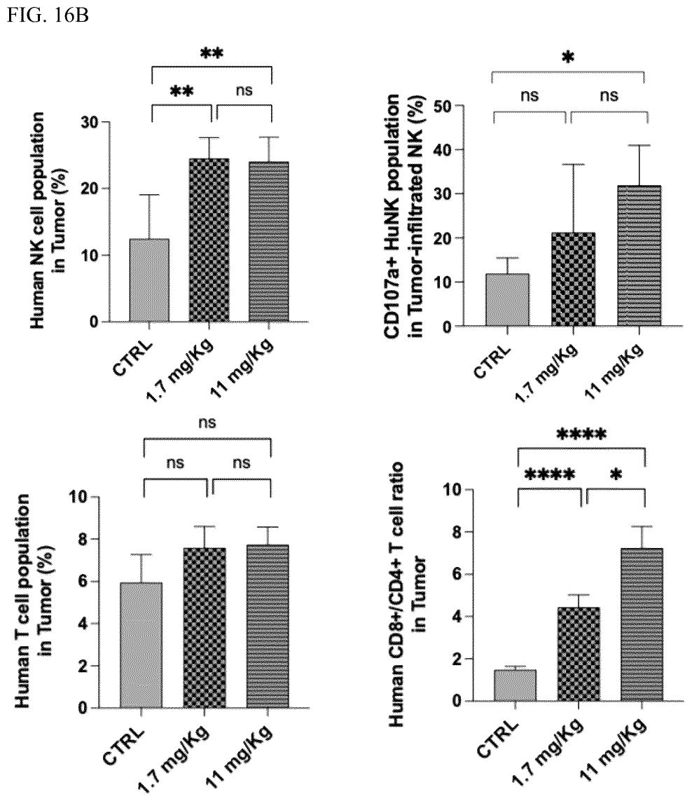

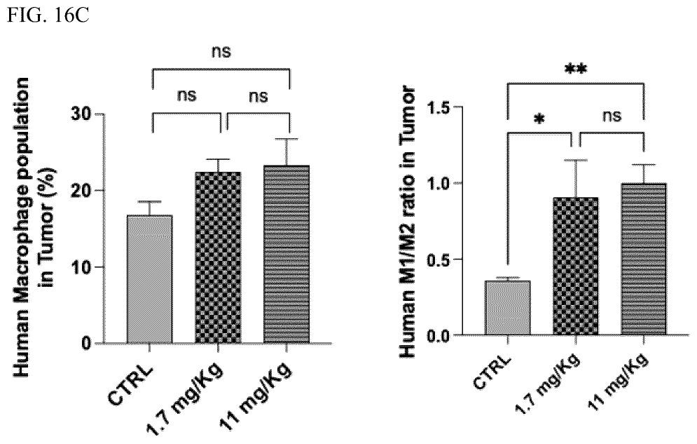

Comprehensive profiling of immune cells in tumor tissue was conducted to characterize the immune response following the administration of the recombinant CAR fusion protein. Tumor tissue was homogenized and dissociated by collagenase to obtain single cells. The single cells were labeled with various antibodies: anti-human CD45 (human hematopoietic marker), anti-human CD56 (human NK cell marker), anti-human CD3 (human T cell marker), anti-human CD4 (helper/regulatory human T cells marker), anti-human CD8 (cytotoxic human T cell marker), anti-human CD68 (human macrophage marker), anti-CD80 (human M1 macrophage marker), and anti-CD206 (human M2 macrophage marker). Administration of the recombinant CAR fusion protein increased the population of tumor-infiltrating human immune cells in the tumor microenvironment (TME) compared to the control group. The recombinant CAR fusion protein specifically modified human NK cells into CAR-NK cells, and the human CAR-NK cells secreted cytokines that reacted with other human immune cells. In addition, both the recombinant CAR fusion protein-injected groups had significantly increased ratios of human CD8+/CD4+ T cells and human M1/M2 macrophages. Therefore, it was confirmed that the recombinant CAR fusion protein induces a comprehensive immune response in the TME. The population of human CD107a+ NK cells in the TME was also increased in both recombinant CAR fusion protein-injected groups. These results are in line with the data confirming the increased population of human CD107a+ NK cells in blood. Taken together, these results indicate that the recombinant CAR fusion protein generates a comprehensive immune response in the TME, accounting for its promising therapeutic efficacy. See FIGS. 16A, 16B and 16C.

### (P) Initial Immunogenicity Test

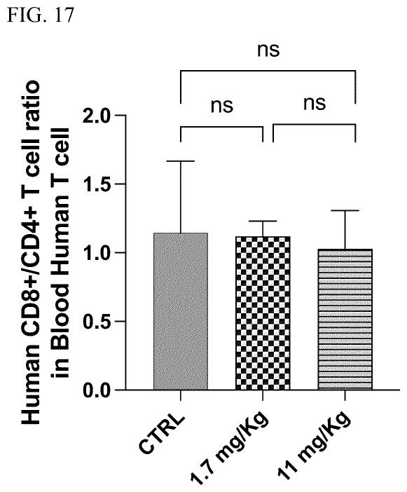

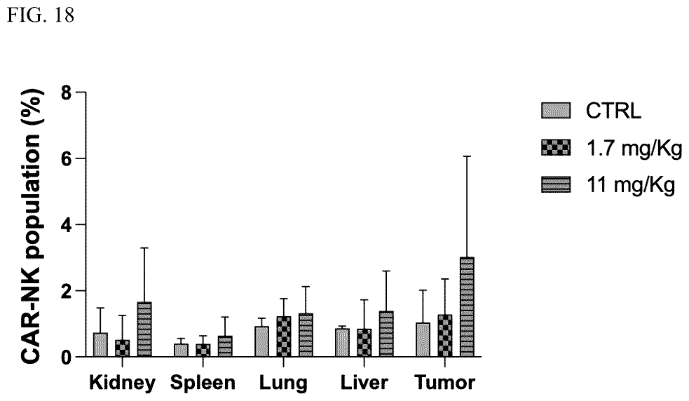

To address the potential immunogenicity of the recombinant CAR fusion protein, the ratio of CD8+/CD4+ human T cells in the blood was analyzed. The immunogenicity reaction would result in the proliferation of CD4+ T cells, thereby changing the ratio of CD8+/CD4+ human T cells. It was found that the recombinant CAR fusion protein-injected groups had insignificant differences in the ratio of CD8+/CD4+ human T cells compared to the control group, indicating that they did not cause immunogenicity. See FIG. 17.

### (Q) In Vivo CAR-NK Biodistribution

To analyze the biodistribution of the in vivo CAR-NK cells, organs including kidney, spleen, lung, liver and tumor were isolated on D+28, which was 7 days after the final in vivo CAR infusion. Tumor tissues were homogenized and dissociated by collagenase to obtain single cells. The cells were fixed and permeabilized to label intracellular his-tag. PE-conjugated anti-his tag antibody was treated to the cells, and the cells were analyzed by flow cytometry. In the 11 mg/Kg recombinant CAR fusion protein-injected group, the population of in vivo CAR-NK cells increased in the tumor tissues compared to the other tissues. This data demonstrates that recombinant CAR fusion protein modifies NK cells into CAR-NK cells in vivo, and reliably directs those cells to the tumor. See FIG. 18.

As discussed above, the recombinant CAR fusion protein targeting PD-L1 expressed on various cancer cells was firstly created and presented in the present disclosure. It was verified that this recombinant CAR-PDL1 protein spontaneously modifies NK cells into CAR-NK PDL1. The resulting CAR-NK PDL1 captures PD-L1 protein (antigen) more efficiently than naïve NK cells, resulting in an increase in the secretion of cytotoxic/cytolytic cytokines upon PD-L1 binding to CAR-PDL1 protein. This result indicates that CAR-NK PDL1 is successfully generated and able to recognize its complementary antigen. It was also verified that CAR-NK PDL1 had more than twice stronger anti-cancer activity and significantly enhanced secretion of cytokines after co-culture of CAR-NK PDL1 with triple-negative breast cancer cells, which express PD-L1. Consequently, it was confirmed that the recombinant CAR fusion protein modifies immune cells into CAR-immune cells and can be developed for both of in vivo and ex vivo applications.

### Example 2

### (A) Preparation of the Recombinant CAR Fusion Protein Targeting PD-L1

A recombinant CAR fusion protein for T cell targeting PD-L1 is designed with mixing the T cell targeting antibody and intracellular stimulatory region from following table.

Table 2 (T cell surface marker for Immune cell-targeting antibody domain and intracellular stimulator for intracellular signal domain of recombinant CAR fusion protein for T cell CAR-T)

| | T cell hinge and | |
|---|---|---|
| | transmembrane | T cell intracellular |
| T cell targeting domain | domain | simulator domain |
| CD3 | CD3 | CD3 ζ |
| CD4 | CD8 | CD28 |
| CD5 | CD28 | 4-1BB |
| CCR7 (CD8+ Cytotoxic T) | IgG4 | CD40 |
| CD107a (CD8+ Cytotoxic T) | | OX-40 |
| CD28 (CD8+ Cytotoxic T) | | CD27 |
| CD45RA (CD8+ Cytotoxic T) | | ICOS |
| CD8 (CD8+ Cytotoxic T) | | DAP12 |
| CD95 (CD8+ Cytotoxic T) | | FcRγ |
| FasL (CD8+ Cytotoxic T) | | CD8 |
| TRAIL (CD8+ Cytotoxic T) | | CD4 |
| 2B4 (Ex-T) | | ITAM |
| BTLA (Ex-T) | | TLR2 |
| CD160 (Ex-T) | | IL2Rb |
| CD226 (Ex-T) | | STAT3-binding motif |
| CD96 (Ex-T) | | DAP10 |
| CTLA4 (Ex-T) | | 2B4 |
| GITR (Ex-T) | | |
| LAG-3 (Ex-T) | | |
| PD-1 (Ex-T) | | |
| TIGIT (Ex-T) | | |
| TIM-3 (Ex-T) | | |
| VISTA (Ex-T) | | |
| TCRγ/δ (γδT) | | |
| IL23 Receptor (γδT) | | |
| TCR Vα24 (NKT) | | |
| TCR Vβ11 (NKT) | | |

The CAR fusion protein for T cells is prepared in the following structure.
Anti-T cell targeting antibody Vh-IgG Hinge-IgG CH2-IgG CH3-linker-Furin cleavage site-membrane targeting-anti-PD-LI Vh-Hinge-Transmembrane region-Intracellular region 1-CD3 zeta-His tag

### (B) Confirmation of the Uptake of the Recombinant CAR Fusion Protein Targeting PD-L1 by T Cells

The recombinant CAR fusion protein targeting PD-L1 is incubated with T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to T cells (1×10⁶ cells/0.5 mL) for 24-hour. Protein transduction is confirmed with same method as discussed under Example 2 (B) above. The result is anticipated that the protein uptake is saturated over 1500 nM as like CAR fusion protein transduction to NK cells, and 1000 nM is selected as the optimal transduction concentration.

### (D) Target (PD-L1) Binding Ability of the Recombinant CAR Fusion Protein

After the CAR-T cells are generated, PD-L1 protein is treated to both CAR-T cells and the T cells and incubated for 2-hour. Because PD-L1 protein is biotinylated, the PD-L1 protein-bound cells can be identified using APC-streptavidin. The cells are not permeabilized at this experiment in order to stain exterior biotin-tag which is conjugated on PD-L1 protein. It is expected that the CAR-T cells have higher APC intensity, which indicates that more PD-L1 protein binds to the CAR-T cells than normal T cells due to the CAR protein-targeting PD-L1.

(E) Cytokine Release from T Cells or CAR-T Cells after PD-L1 Binding Through PD-L1 Protein Treatment.

In order to verify the increased anti-cancer activity owing to the CAR fusion protein, cytotoxic/cytolytic cytokine secretion upon PD-L1 binding to CAR-T and T cells is analyzed. After PD-L1 binding to the T cells or CAR-T cells (2-hour treatment of 20 ng/ml PD-L1 protein), cell supernatant is harvested and ran cytokine ELISA as like CAR-NK cell analysis. It is anticipated that cytokine secretion of the CAR-T cells is overly increased as compared to that of the T cells. This result implies that the CAR-T cells have stronger anti-cancer activity over the T cells upon the binding with antigen.

### (F) Anti-Cancer Activity of T Cell: Cancer Cell Death

Anti-cancer activity of the CAR-T cells and T cells are compared through induction of cancer cell death. The experiment condition is as follows.
CellTracker Blue™ labelled MDA-MB-231 cancer cells
Effector:Target (E:T)=5:1
4-hour co-incubation of CellTracker Blue™ labelled-MDA-MB-231 & T cells/CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-T cells have stronger anti-cancer activity as compared to the T cells.

### (G) Anti-Cancer Activity of T Cell:T Cells' Cytokines

Cytokine secretion from the T cells and the CAR-T cells are measured from the cell supernatant after 4 hours co-incubation of cancer cells with the T cells or the CAR-T cells. The result is expected that cytotoxic/cytolytic cytokines are more secreted from and the CAR-T cells upon cancer cell engraftment than the T cells. The cytokine result is anticipated to have correlation with cancer cell death to demonstrate that the increased cancer cell death of the CAR-T cells are induced by the increased cytokine secretion. Consequently, it is anticipated that the recombinant CAR fusion protein can modify T cells to have a CAR system without viral vector-mediated gene transfection and genetic expression time, and consequently, bring the stronger anti-cancer activity.

### Example 3

### (A) Preparation of the Recombinant CAR Fusion Protein Targeting PD-L1

A recombinant CAR fusion protein for both NK and T cell targeting PD-L1 is designed with mixing the antibody, simultaneously targeting NK and T cell, and intracellular stimulatory region.

The anti-PD-L1 CAR fusion protein for both NK and T cells is prepared in the following structure.

Anti-NK&T cell targeting antibody Vh-IgG Hinge-IgG CH2-IgG
CH3-linker-Furin cleavage site-membrane targeting-anti-PD-L1 Vh-
Hinge-Transmembrane region-Intracellular region 1-CD3 zeta-His tag

| | NK&T cell | NK&T cell |
|---|---|---|
| NK&T cell | hinge and | intracellular |
| targeting | transmembrane | stimulator |
| domain | domain | domain |
| CD4 | CD3 | CD3 ζ |
| CCR7 | CD8 | CD28 |
| CD107a | CD28 | 4-1BB |
| CD28 | 2B4 | CD40 |
| CD45RA | 4-1BB | OX-40 |
| CD8 | IgG4 | CD27 |
| FasL | | ICOS |
| TRAIL | | DAP12 |
| 2B4 | | FcRγ |
| BTLA | | CD8 |
| CD160 | | CD4 |
| CD226 | | ITAM |
| CD96 | | TLR2 |
| CTLA4 | | IL2Rb |
| GITR | | STAT3-binding motif |
| LAG-3 | | DAP10 |
| PD-1 | | 2B4 |
| TIGIT | | NKp30 |
| TIM-3 | | NKG2D |
| VISTA | | |
| IL23 Receptor | | |
| CD161 | | |
| NKG2D | | |
| NKG2A | | |
| NKp30 | | |
| CD7 | | |

### (B) Confirmation of the Uptake of the Recombinant CAR Fusion Protein Targeting PD-L1 by NK and T Cells

The recombinant CAR fusion protein targeting PD-L1 is incubated with NK and T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK and T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK and T cells (1×10⁶ cells/0.5 mL) for 24-hour. Protein transduction is confirmed with same method as discussed under Example (B) above. The result is anticipated that the protein uptake is saturated over 1500 nM as like CAR-PD-L1 fusion protein transduction to NK cells, and 1000 nM is selected as the optimal transduction concentration.

### (D) Target (PD-L1) Binding Ability of the Recombinant CAR Fusion Protein

After the CAR-NK and CAR-T cells are generated, PD-L1 protein is treated to CAR-NK cells, CAR-T cells, NK cells and the T cells and incubated for 2-hour. Because PD-L1 protein is biotinylated, the PD-L1 protein-bound cells can be identified using APC-streptavidin. The cells are not permeabilized at this experiment in order to stain exterior biotin-tag which is conjugated on PD-L1 protein. It is expected that the CAR-NK cells and CAR-T cells have higher APC intensity, which indicates that more PD-L1 protein binds to the CAR-NK cells and CAR-T cells than normal NK cells and T cells due to the CAR protein-targeting PD-L1.

(E) Cytokine Release from NK Cells, T Cells CAR-NK Cells, or CAR-T Cells after PD-L1 Binding Through PD-L1 Protein Treatment.

In order to verify the increased anti-cancer activity owing to the CAR fusion protein, cytotoxic/cytolytic cytokine secretion upon PD-L1 binding to CAR-NK cells, CAR-T cells, NK cells and T cells is analyzed. After PD-L1 binding to the cells (2-hour treatment of 20 ng/ml PD-L1 protein), cell supernatant is harvested and ran cytokine ELISA. It is anticipated that cytokine secretions of the CAR-NK cells and CAR-T cells are overly increased as compared to that of the NK cells and T cells. This result implies that the CAR-NK cells and CAR-T cells have stronger anti-cancer activity over normal NK cells and T cells upon the binding with antigen.

### (F) Anti-Cancer Activity of CAR-NK and CAR-T Cell: Cancer Cell Death

Anti-cancer activity of the combination of CAR-NK cells and CAR-T cells and another combination of NK cells and T cells are compared through induction of cancer cell death. The experiment condition is as follows.
CellTracker Blue™ labelled MDA-MB-231 cancer cells
Effector:Target (E:T)=5:1
4-hour co-incubation of CellTracker Blue™ labelled-MDA-MB-231 with NK&T cells/CAR-NK&CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the combination of CAR-NK cells and CAR-T cells have stronger anti-cancer activity as compared to the combination of NK cells and T cells.

### (G) Anti-Cancer Activity of CAR-NK Cells and CAR-T Cell: Cytokine Secretion

The secreted cytokines are measured from the cell supernatant after 4 hours co-incubation of cancer cells with the combination of NK cells and T cells or the other combination of CAR-NK cells and CAR-T cells. The result is expected that cytotoxic/cytolytic cytokines are more secreted from and the combination of CAR-NK cells and CAR-T cells upon cancer cell engraftment than the combination of NK cells and T cells. The cytokine result is anticipated to have correlation with cancer cell death to demonstrate that the increased cancer cell death from the combination group of CAR-NK cells and CAR-T cells is induced by the increased cytokine secretion. Consequently, it is anticipated that the recombinant CAR fusion protein can simultaneously modify NK cells and T cells to have a CAR system without viral vector-mediated gene transfection and genetic expression time, and consequently, bring the stronger anti-cancer activity.

### Example 4

### (A) Preparation of the Recombinant CAR Fusion Protein Targeting CD19

A recombinant CAR fusion protein for NK cell targeting CD19 is designed with mixing the NK cell targeting antibody and intracellular stimulatory region.

The anti-CD19 CAR fusion protein for NK cells is prepared in the following structure.

Anti-NK cell targeting antibody Vh-IgG Hinge-IgG CH2-IgG CH3-linker-Furin cleavage site-membrane targeting-anti-CD19 Vh-Hinge-Transmembrane region-Intracellular region 1-CD3 zeta-His tag

### (B) Confirmation of the Uptake of the Recombinant CAR Fusion Protein Targeting CD19 by NK Cells

The recombinant CAR fusion protein targeting CD19 is incubated with NK cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK cells (1×10⁶ cells/0.5 mL) for 24-hour. Protein transduction is confirmed with same method as discussed under Example 2 (B) above. The result is anticipated that the protein uptake is saturated over 1500 nM as like CAR-PD-L1 fusion protein transduction to NK cells, and 1000 nM is selected as the optimal transduction concentration.

### (D) Target (CD19) Binding Ability of the Recombinant CAR Fusion Protein

After the CAR-NK cells are generated, CD19 protein is treated to both CAR-NK cells and the NK cells and incubated for 2-hour. Because CD19 protein is biotinylated, the CD19 protein-bound cells can be identified using APC-streptavidin. The cells are not permeabilized at this experiment in order to stain exterior biotin-tag which is conjugated on CD19 protein. It is expected that the CAR-NK cells have higher APC intensity, which indicates that more CD19 protein binds to the CAR-NK cells than normal NK cells due to the CAR protein-targeting CD19.

(E) Cytokine Release from NK Cells or CAR-NK Cells after CD19 Binding Through CD19 Protein Treatment.

In order to verify the increased anti-cancer activity owing to the CAR fusion protein, cytotoxic/cytolytic cytokine secretion upon CD19 binding to CAR-NK and NK cells is analyzed. After CD19 binding to the NK cells or CAR-NK cells (2-hour treatment of 20 ng/mL CD19 protein), cell supernatant is harvested and ran cytokine ELISA. It is anticipated that cytokine secretion of the CAR-NK cells is overly increased as compared to that of the NK cells. This result implies that the CAR-NK cells have stronger anti-cancer activity over the NK cells upon the binding with antigen.

### (F) Anti-Cancer Activity of NK Cell: Cancer Cell Death

Anti-cancer activity of the CAR-NK cells and NK cells are compared through induction of cancer cell death. The experiment condition is as follows.
CellTracker Blue™ labelled NALM6 cancer cells
Effector:Target (E:T)=5:1
4-hour co-incubation of CellTracker Blue™ labelled-NALM6 & NK cells/CAR-NK cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-NK cells have stronger anti-cancer activity as compared to the NK cells.

### (G) Anti-Cancer Activity of NK Cell: NK Cells' Cytokines

Cytokine secretion from the NK cells and the CAR-NK cells are measured from the cell supernatant after 4 hours co-incubation of cancer cells with the NK cells or the CAR-NK cells. The result is expected that cytotoxic/cytolytic cytokines are more secreted from and the CAR-NK cells upon cancer cell engraftment than the NK cells. The cytokine result is anticipated to have correlation with cancer cell death to demonstrate that the increased cancer cell death of the CAR-NK cells is induced by the increased cytokine secretion. Consequently, it is anticipated that the recombinant CAR fusion protein can modify NK cells to have a CAR system without viral vector-mediated gene transfection and genetic expression time, and consequently, bring the stronger anti-cancer activity.

### Example 5

### (A) Preparation of the Recombinant CAR Fusion Protein Targeting CD19

A recombinant CAR fusion protein for T cell targeting CD19 is designed with mixing the T cell targeting antibody and intracellular stimulatory region from Table2.

The anti-CD19 CAR fusion protein for T cells is prepared in the following structure.

Anti-T cell targeting antibody Vh-IgG Hinge-IgG CH2-IgG CH3-linker-Furin cleavage site-membrane targeting-anti-CD19 Vh-Hinge-Transmembrane region-Intracellular region 1-CD3 zeta-His tag

(B) Confirmation of the Uptake of the Recombinant CAR Fusion Protein Targeting CD19 by T cells

The recombinant CAR fusion protein targeting CD19 is incubated with T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to T cells (1×10⁶ cells/0.5 mL) for 24-hour. Protein transduction is confirmed with same method as discussed under Example 2 (B) above. The result is anticipated that the protein uptake is saturated over 1500 nM as like CAR-PD-L1 fusion protein transduction to NK cells, and 1000 nM is selected as the optimal transduction concentration.

### (D) Target (CD19) Binding Ability of the Recombinant CAR Fusion Protein

After the CAR-T cells are generated, CD19 protein is treated to both CAR-T cells and the T cells and incubated for 2-hour. Because CD19 protein is biotinylated, the CD19 protein-bound cells can be identified using APC-streptavidin. The cells are not permeabilized at this experiment in order to stain exterior biotin-tag which is conjugated on CD19 protein. It is expected that the CAR-T cells have higher APC intensity, which indicates that more CD19 protein binds to the CAR-T cells than normal T cells due to the CAR protein-targeting CD19.

(E) Cytokine Release from T Cells or CAR-T Cells after CD19 Binding Through CD19 Protein Treatment.

In order to verify the increased anti-cancer activity owing to the CAR fusion protein, cytotoxic/cytolytic cytokine secretion upon CD19 binding to CAR-T and T cells is analyzed. After CD19 binding to the T cells or CAR-T cells (2-hour treatment of 20 ng/mL CD19 protein), cell supernatant is harvested and ran cytokine ELISA as like CAR-NK cell analysis. It is anticipated that cytokine secretion of the CAR-T cells is overly increased as compared to that of the T cells. This result implies that the CAR-T cells have stronger anti-cancer activity over the T cells upon the binding with antigen.

### (F) Anti-Cancer Activity of T Cell: Cancer Cell Death

Anti-cancer activity of the CAR-T cells and T cells are compared through induction of cancer cell death. The experiment condition is as follows.
CellTracker Blue™ labelled NALM6 cancer cells
Effector:Target (E:T)=5:1
4-hour co-incubation of CellTracker Blue™ labelled-NALM6 & T cells/CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-T cells have stronger anti-cancer activity as compared to the T cells.

### (G) Anti-Cancer Activity of T Cell:T Cells' Cytokines

Cytokine secretion from the T cells and the CAR-T cells are measured from the cell supernatant after 4 hours co-incubation of cancer cells with the T cells or the CAR-T cells. The result is expected that cytotoxic/cytolytic cytokines are more secreted from and the CAR-T cells upon cancer cell engraftment than the T cells. The cytokine result is anticipated to have correlation with cancer cell death to demonstrate that the increased cancer cell death of the CAR-T cells is induced by the increased cytokine secretion. Consequently, it is anticipated that the recombinant CAR fusion protein can modify T cells to have a CAR system without viral vector-mediated gene transfection and genetic expression time, and consequently, bring the stronger anti-cancer activity.

### Example 6

### (A) Preparation of the Recombinant CAR Fusion Protein Targeting CD19

A recombinant CAR fusion protein for both NK and T cell targeting CD19 is designed with mixing the antibody, simultaneously targeting NK and T cell, and intracellular stimulatory region.

The anti-CD19 CAR fusion protein for both NK and T cells is prepared in the following structure.

Anti-NK&T cell targeting antibody Vh-IgG Hinge-IgG CH2-IgG CH3-linker-Furin cleavage site-membrane targeting-anti-CD19 Vh-Hinge-Transmembrane region-Intracellular region 1-CD3 zeta-His tag

### (B) Confirmation of the Uptake of the Recombinant CAR Fusion Protein Targeting CD19 by NK and T Cells

The recombinant CAR fusion protein targeting CD19 is incubated with NK and T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK and T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK and T cells (1×10⁶ cells/0.5 mL) for 24-hour. Protein transduction is confirmed with same method as discussed under Example (B) above. The result is anticipated that the protein uptake is saturated over 1500 nM as like CAR-PD-L1 fusion protein transduction to NK cells, and 1000 nM is selected as the optimal transduction concentration.

### (D) Target (CD19) Binding Ability of the Recombinant CAR Fusion Protein

After the CAR-NK and CAR-T cells are generated, CD19 protein is treated to CAR-NK cells, CAR-T cells, NK cells and the T cells and incubated for 2-hour. Because CD19 protein is biotinylated, the CD19 protein-bound cells can be identified using APC-streptavidin. The cells are not permeabilized at this experiment in order to stain exterior biotin-tag which is conjugated on CD19 protein. It is expected that the CAR-NK cells and CAR-T cells have higher APC intensity, which indicates that more CD19 protein binds to the CAR-NK cells and CAR-T cells than normal NK cells and T cells due to the CAR protein-targeting CD19.

(E) Cytokine Release from NK Cells, T Cells CAR-NK Cells, or CAR-T Cells after CD19 Binding Through CD19 Protein Treatment.

In order to verify the increased anti-cancer activity owing to the CAR fusion protein, cytotoxic/cytolytic cytokine secretion upon CD19 binding to CAR-NK cells, CAR-T cells, NK cells and T cells is analyzed. After CD19 binding to the cells (2-hour treatment of 20 ng/mL CD19 protein), cell supernatant is harvested and ran cytokine ELISA. It is anticipated that cytokine secretions of the CAR-NK cells and CAR-T cells are overly increased as compared to that of the NK cells and T cells. This result implies that the CAR-NK cells and CAR-T cells have stronger anti-cancer activity over normal NK cells and T cells upon the binding with antigen.

### (F) Anti-Cancer Activity of CAR-NK and CAR-T Cell: Cancer Cell Death

Anti-cancer activity of the combination of CAR-NK cells and CAR-T cells and another combination of NK cells and T cells are compared through induction of cancer cell death. The experiment condition is as follows.
CellTracker Blue™ labelled NAML6 cancer cells
Effector:Target (E:T)=5:1.
4-hour co-incubation of CellTracker Blue™ labelled-NAML6 with NK&T cells/CAR-NK&CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the combination of CAR-NK cells and CAR-T cells have stronger anti-cancer activity as compared to the combination of NK cells and T cells.

### (G) Anti-Cancer Activity of CAR-NK Cells and CAR-T Cell: Cytokine Secretion

The secreted cytokines are measured from the cell supernatant after 4 hours co-incubation of cancer cells with the combination of NK cells and T cells or the other combination of CAR-NK cells and CAR-T cells. The result is expected that cytotoxic/cytolytic cytokines are more secreted from and the combination of CAR-NK cells and CAR-T cells upon cancer cell engraftment than the combination of NK cells and T cells. The cytokine result is anticipated to have correlation with cancer cell death to demonstrate that the increased cancer cell death from the combination group of CAR-NK cells and CAR-T cells is induced by the increased cytokine secretion. Consequently, it is anticipated that the recombinant CAR fusion protein can simultaneously modify NK cells and T cells to have a CAR system without viral vector-mediated gene transfection and genetic expression time, and consequently, bring the stronger anti-cancer activity.

### Example 7

### (A) Preparation of the Recombinant CAR Fusion Protein Targeting HER2

A recombinant CAR fusion protein for NK cell targeting HER2 is designed with mixing the NK cell targeting antibody and intracellular stimulatory region.

The anti-HER2 CAR fusion protein for NK cells is prepared in the following structure.

Anti-NK cell targeting antibody Vh-IgG Hinge-IgG CH2-IgG CH3-linker-Furin cleavage site-membrane targeting-anti-HER2 Vh-Hinge-Transmembrane region-Intracellular region 1-CD3 zeta-His tag

### (B) Confirmation of the Uptake of the Recombinant CAR Fusion Protein Targeting HER2 by NK Cells

The recombinant CAR fusion protein targeting HER2 is incubated with NK cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK cells (1×10⁶ cells/0.5 mL) for 24-hour. Protein transduction is confirmed with same method as discussed under Example 2 (B) above. The result is anticipated that the protein uptake is saturated over 1500 nM as like CAR-PD-L1 fusion protein transduction to NK cells, and 1000 nM is selected as the optimal transduction concentration.

### (D) Target (HER2) Binding Ability of the Recombinant CAR Fusion Protein

After the CAR-NK cells are generated, HER2 protein is treated to both CAR-NK cells and the NK cells and incubated for 2-hour. Because HER2 protein is biotinylated, the HER2 protein-bound cells can be identified using APC-streptavidin. The cells are not permeabilized at this experiment in order to stain exterior biotin-tag which is conjugated on HER2 protein. It is expected that the CAR-NK cells have higher APC intensity, which indicates that more HER2 protein binds to the CAR-NK cells than normal NK cells due to the CAR protein-targeting HER2.

(E) Cytokine Release from NK Cells or CAR-NK Cells after HER2 Binding Through HER2 Protein Treatment.

In order to verify the increased anti-cancer activity owing to the CAR fusion protein, cytotoxic/cytolytic cytokine secretion upon HER2 binding to CAR-NK and NK cells is analyzed. After HER2 binding to the NK cells or CAR-NK cells (2-hour treatment of 20 ng/mL HER2 protein), cell supernatant is harvested and ran cytokine ELISA. It is anticipated that cytokine secretion of the CAR-NK cells is overly increased as compared to that of the NK cells. This result implies that the CAR-NK cells have stronger anti-cancer activity over the NK cells upon the binding with antigen.

### (F) Anti-Cancer Activity of NK Cell: Cancer Cell Death

Anti-cancer activity of the CAR-NK cells and NK cells are compared through induction of cancer cell death. The experiment condition is as follows.
CellTracker Blue™ labelled BT-474 cancer cells
Effector:Target (E:T)=5:1
4-hour co-incubation of CellTracker Blue™ labelled-BT-474 & NK cells/CAR-NK cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-NK cells have stronger anti-cancer activity as compared to the NK cells.

### (G) Anti-Cancer Activity of NK Cell: NK Cells' Cytokines

Cytokine secretion from the NK cells and the CAR-NK cells are measured from the cell supernatant after 4 hours co-incubation of cancer cells with the NK cells or the CAR-NK cells. The result is expected that cytotoxic/cytolytic cytokines are more secreted from and the CAR-NK cells upon cancer cell engraftment than the NK cells. The cytokine result is anticipated to have correlation with cancer cell death to demonstrate that the increased cancer cell death of the CAR-NK cells is induced by the increased cytokine secretion. Consequently, it is anticipated that the recombinant CAR fusion protein can modify NK cells to have a CAR system without viral vector-mediated gene transfection and genetic expression time, and consequently, bring the stronger anti-cancer activity.

### Example 8

### (A) Preparation of the Recombinant CAR Fusion Protein Targeting HER2

A recombinant CAR fusion protein for T cell targeting HER2 is designed with mixing the T cell targeting antibody and intracellular stimulatory region from Table2.

The anti-HER2 CAR fusion protein for T cells is prepared in the following structure.

Anti-T cell targeting antibody Vh-IgG Hinge-IgG CH2-IgG CH3-linker-Furin cleavage site-membrane targeting-anti-HER2 Vh-Hinge-Transmembrane region-Intracellular region 1-CD3 zeta-His tag

### (B) Confirmation of the Uptake of the Recombinant CAR Fusion Protein Targeting HER2 by T Cells

The recombinant CAR fusion protein targeting HER2 is incubated with T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to T cells (1×10⁶ cells/0.5 mL) for 24-hour. Protein transduction is confirmed with same method as discussed under Example 2 (B) above. The result is anticipated that the protein uptake is saturated over 1500 nM as like CAR-PD-L1 fusion protein transduction to NK cells, and 1000 nM is selected as the optimal transduction concentration.

### (D) Target (HER2) Binding Ability of the Recombinant CAR Fusion Protein

After the CAR-T cells are generated, HER2 protein is treated to both CAR-T cells and the T cells and incubated for 2-hour. Because HER2 protein is biotinylated, the HER2 protein-bound cells can be identified using APC-streptavidin. The cells are not permeabilized at this experiment in order to stain exterior biotin-tag which is conjugated on HER2 protein. It is expected that the CAR-T cells have higher APC intensity, which indicates that more HER2 protein binds to the CAR-T cells than normal T cells due to the CAR protein-targeting HER2.

(E) Cytokine Release from T Cells or CAR-T Cells after HER2 Binding Through HER2 Protein Treatment.

In order to verify the increased anti-cancer activity owing to the CAR fusion protein, cytotoxic/cytolytic cytokine secretion upon HER2 binding to CAR-T and T cells is analyzed. After HER2 binding to the T cells or CAR-T cells (2-hour treatment of 20 ng/ml HER2 protein), cell supernatant is harvested and ran cytokine ELISA. It is anticipated that cytokine secretion of the CAR-T cells is overly increased as compared to that of the T cells. This result implies that the CAR-T cells have stronger anti-cancer activity over the T cells upon the binding with antigen.

### (F) Anti-Cancer Activity of T Cell: Cancer Cell Death

Anti-cancer activity of the CAR-T cells and T cells are compared through induction of cancer cell death. The experiment condition is as follows.
CellTracker Blue™ labelled BT-474 cancer cells
Effector:Target (E:T)=5:1.
4-hour co-incubation of CellTracker Blue™ labelled BT-474 & T cells/CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-T cells have stronger anti-cancer activity as compared to the T cells.

### (G) Anti-Cancer Activity of T Cell: T Cells' Cytokines

Cytokine secretion from the T cells and the CAR-T cells are measured from the cell supernatant after 4 hours co-incubation of cancer cells with the T cells or the CAR-T cells. The result is expected that cytotoxic/cytolytic cytokines are more secreted from and the CAR-T cells upon cancer cell engraftment than the T cells. The cytokine result is anticipated to have correlation with cancer cell death to demonstrate that the increased cancer cell death of the CAR-T cells is induced by the increased cytokine secretion. Consequently, it is anticipated that the recombinant CAR fusion protein can modify T cells to have a CAR system without viral vector-mediated gene transfection and genetic expression time, and consequently, bring the stronger anti-cancer activity.

### Example 9

### (A) Preparation of the Recombinant CAR Fusion Protein Targeting HER2

A recombinant CAR fusion protein for both NK and T cell targeting HER2 is designed with mixing the antibody, simultaneously targeting NK and T cell, and intracellular stimulatory region.

The anti-HER2 CAR fusion protein for both NK and T cells is prepared in the following structure.

Anti-NK&T cell targeting antibody Vh-IgG Hinge-IgG CH2-IgG CH3-linker-Furin cleavage site-membrane targeting-anti-HER2 Vh-Hinge-Transmembrane region-Intracellular region 1-CD3 zeta-His tag

### (B) Confirmation of the Uptake of the Recombinant CAR Fusion Protein Targeting HER2 by NK and T Cells

The recombinant CAR fusion protein targeting HER2 is incubated with NK and T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK and T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK and T cells (1×10⁶ cells/0.5 mL) for 24-hour. Protein transduction is confirmed with same method as discussed under Example (B) above. The result is anticipated that the protein uptake is saturated over 1500 nM as like CAR-PD-L1 fusion protein transduction to NK cells, and 1000 nM is selected as the optimal transduction concentration.

### (D) Target (HER2) Binding Ability of the Recombinant CAR Fusion Protein

After the CAR-NK and CAR-T cells are generated, HER2 protein is treated to CAR-NK cells, CAR-T cells, NK cells and the T cells and incubated for 2-hour. Because HER2 protein is biotinylated, the HER2 protein-bound cells can be identified using APC-streptavidin. The cells are not permeabilized at this experiment in order to stain exterior biotin-tag which is conjugated on HER2 protein. It is expected that the CAR-NK cells and CAR-T cells have higher APC intensity, which indicates that more HER2 protein binds to the CAR-NK cells and CAR-T cells than normal NK cells and T cells due to the CAR protein-targeting HER2.

(E) Cytokine Release from NK Cells, T Cells CAR-NK Cells, or CAR-T Cells after HER2 Binding Through HER2 Protein Treatment.

In order to verify the increased anti-cancer activity owing to the CAR fusion protein, cytotoxic/cytolytic cytokine secretion upon HER2 binding to CAR-NK cells, CAR-T cells, NK cells and T cells is analyzed. After HER2 binding to the cells (2-hour treatment of 20 ng/mL HER2 protein), cell supernatant is harvested and ran cytokine ELISA. It is anticipated that cytokine secretions of the CAR-NK cells and CAR-T cells are overly increased as compared to that of the NK cells and T cells. This result implies that the CAR-NK cells and CAR-T cells have stronger anti-cancer activity over normal NK cells and T cells upon the binding with antigen.

### (F) Anti-Cancer Activity of CAR-NK and CAR-T Cell: Cancer Cell Death

Anti-cancer activity of the combination of CAR-NK cells and CAR-T cells and another combination of NK cells and T cells are compared through induction of cancer cell death. The experiment condition is as follows.
CellTracker Blue™ labelled BT-474 cancer cells
Effector:Target (E:T)=5:1.
4-hour co-incubation of CellTracker Blue™ labelled-BT-474 with NK&T cells/CAR-NK&CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the combination of CAR-NK cells and CAR-T cells have stronger anti-cancer activity as compared to the combination of NK cells and T cells.

### (G) Anti-Cancer Activity of CAR-NK Cells and CAR-T Cell: Cytokine Secretion

The secreted cytokines are measured from the cell supernatant after 4 hours co-incubation of cancer cells with the combination of NK cells and T cells or the other combination of CAR-NK cells and CAR-T cells. The result is expected that cytotoxic/cytolytic cytokines are more secreted from and the combination of CAR-NK cells and CAR-T cells upon cancer cell engraftment than the combination of NK cells and T cells. The cytokine result is anticipated to have correlation with cancer cell death to demonstrate that the increased cancer cell death from the combination group of CAR-NK cells and CAR-T cells is induced by the increased cytokine secretion. Consequently, it is anticipated that the recombinant CAR fusion protein can simultaneously modify NK cells and T cells to have a CAR system without viral vector-mediated gene transfection and genetic expression time, and consequently, bring the stronger anti-cancer activity.

### Example 10

### (A) Preparation of the Recombinant CAR Fusion Protein Targeting TROP2

A recombinant CAR fusion protein for NK cell targeting TROP2 is designed with mixing the NK cell targeting antibody and intracellular stimulatory region.

The anti-TROP2 CAR fusion protein for NK cells is prepared in the following structure.

Anti-NK cell targeting antibody Vh-IgG Hinge-IgG CH2-IgG CH3-linker-Furin cleavage site-membrane targeting-anti-TROP2 Vh-Hinge-Transmembrane region-Intracellular region 1-CD3 zeta-His tag

### (B) Confirmation of the Uptake of the Recombinant CAR Fusion Protein Targeting TROP2 by NK Cells

The recombinant CAR fusion protein targeting TROP2 is incubated with NK cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the NK cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to NK cells (1×10⁶ cells/0.5 mL) for 24-hour. Protein transduction is confirmed with same method as discussed under Example 2 (B) above. The result is anticipated that the protein uptake is saturated over 1500 nM as like CAR-PD-L1 fusion protein transduction to NK cells, and 1000 nM is selected as the optimal transduction concentration.

### (D) Target (TROP2) Binding Ability of the Recombinant CAR Fusion Protein

After the CAR-NK cells are generated, TROP2 protein is treated to both CAR-NK cells and the NK cells and incubated for 2-hour. Because TROP2 protein is biotinylated, the TROP2 protein-bound cells can be identified using APC-streptavidin. The cells are not permeabilized at this experiment in order to stain exterior biotin-tag which is conjugated on TROP2 protein. It is expected that the CAR-NK cells have higher APC intensity, which indicates that more TROP2 protein binds to the CAR-NK cells than normal NK cells due to the CAR protein-targeting TROP2.

(E) Cytokine Release from NK Cells or CAR-NK Cells after TROP2 Binding Through TROP2 Protein Treatment.

In order to verify the increased anti-cancer activity owing to the CAR fusion protein, cytotoxic/cytolytic cytokine secretion upon TROP2 binding to CAR-NK and NK cells is analyzed. After TROP2 binding to the NK cells or CAR-NK cells (2-hour treatment of 20 ng/mL TROP2 protein), cell supernatant is harvested and ran cytokine ELISA. It is anticipated that cytokine secretion of the CAR-NK cells is overly increased as compared to that of the NK cells. This result implies that the CAR-NK cells have stronger anti-cancer activity over the NK cells upon the binding with antigen.

### (F) Anti-Cancer Activity of NK Cell: Cancer Cell Death

Anti-cancer activity of the CAR-NK cells and NK cells are compared through induction of cancer cell death. The experiment condition is as follows.
CellTracker Blue™ labelled HCC1500 cancer cells
Effector:Target (E:T)=5:1
4-hour co-incubation of CellTracker Blue™ labelled-HCC1500 & NK cells/CAR-NK cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-NK cells have stronger anti-cancer activity as compared to the NK cells.

### (G) Anti-Cancer Activity of NK Cell: NK Cells' Cytokines

Cytokine secretion from the NK cells and the CAR-NK cells are measured from the cell supernatant after 4 hours co-incubation of cancer cells with the NK cells or the CAR-NK cells. The result is expected that cytotoxic/cytolytic cytokines are more secreted from and the CAR-NK cells upon cancer cell engraftment than the NK cells. The cytokine result is anticipated to have correlation with cancer cell death to demonstrate that the increased cancer cell death of the CAR-NK cells is induced by the increased cytokine secretion. Consequently, it is anticipated that the recombinant CAR fusion protein can modify NK cells to have a CAR system without viral vector-mediated gene transfection and genetic expression time, and consequently, bring the stronger anti-cancer activity.

### Example 11

### (A) Preparation of the Recombinant CAR Fusion Protein Targeting TROP2

A recombinant CAR fusion protein for T cell targeting TROP2 is designed with mixing the T cell targeting antibody and intracellular stimulatory region from Table2.

The anti-TROP2 CAR fusion protein for T cells is prepared in the following structure.

Anti-T cell targeting antibody Vh-IgG Hinge-IgG CH2-IgG CH3-linker-Furin cleavage site-membrane targeting-anti-TROP2 Vh-Hinge-Transmembrane region-Intracellular region 1-CD3 zeta-His tag

### (B) Confirmation of the Uptake of the Recombinant CAR Fusion Protein Targeting CD19 by T Cells

The recombinant CAR fusion protein targeting TROP2 is incubated with T cells (1 μM of the CAR fusion protein, 1×10⁶ cells/0.5 mL) for 24-hour. The cells are collected and fixed to be permeabilized. Then, PE-conjugated anti-his tag antibody is treated to label his-tag on the recombinant CAR fusion protein. It is expected that the T cells treated with the recombinant CAR fusion protein show higher PE intensity due to its his-tag.

### (C) The Recombinant CAR Fusion Protein Transduction Optimization

To optimize concentration for the recombinant CAR fusion protein transduction, the recombinant CAR fusion protein with various concentration is treated to T cells (1×10⁶ cells/0.5 mL) for 24-hour. Protein transduction is confirmed with same method as discussed under Example 2 (B) above. The result is anticipated that the protein uptake is saturated over 1500 nM as like CAR fusion protein transduction to NK cells, and 1000 nM is selected as the optimal transduction concentration.

### (D) Target (TROP2) Binding Ability of the Recombinant CAR Fusion Protein

After the CAR-T cells are generated, TROP2 protein is treated to both CAR-T cells and the T cells and incubated for 2-hour. Because TROP2 protein is biotinylated, the TROP2 protein-bound cells can be identified using APC-streptavidin. The cells are not permeabilized at this experiment in order to stain exterior biotin-tag which is conjugated on TROP2 protein. It is expected that the CAR-T cells have higher APC intensity, which indicates that more TROP2 protein binds to the CAR-T cells than normal T cells due to the CAR protein-targeting TROP2.

(E) Cytokine Release from T Cells or CAR-T Cells after TROP2 Binding Through TROP2 Protein Treatment.

In order to verify the increased anti-cancer activity owing to the CAR fusion protein, cytotoxic/cytolytic cytokine secretion upon TROP2 binding to CAR-T and T cells is analyzed. After TROP2 binding to the T cells or CAR-T cells (2-hour treatment of 20 ng/ml CD19 protein), cell supernatant is harvested and ran cytokine ELISA. It is anticipated that cytokine secretion of the CAR-T cells is overly increased as compared to that of the T cells. This result implies that the CAR-T cells have stronger anti-cancer activity over the T cells upon the binding with antigen.

### (F) Anti-Cancer Activity of T Cell: Cancer Cell Death

Anti-cancer activity of the CAR-T cells and T cells are compared through induction of cancer cell death. The experiment condition is as follows.
CellTracker Blue™ labelled HCC1500 cancer cells
Effector:Target (E:T)=5:1
4-hour co-incubation of CellTracker Blue™ labelled-HCC1500 & T cells/CAR-T cells

After co-incubation, cells are harvested and stained Alexa Fluor-488 Annexin V to label dead cells. The cells are run through flow cytometry, and the population of CellTracker Blue™ labelled cells (cancer cells) are analyzed the intensity of Alexa Fluor 488. Double positive of CellTracker Blue™ and Alexa Fluor 488-Annexin V stained cells indicate dead cancer cells. The more population of Alexa Fluor 488-stained cells, the more dead cancer cells. It is predicted that the CAR-T cells have stronger anti-cancer activity as compared to the T cells.

### (G) Anti-Cancer Activity of T Cell: T Cells' Cytokines

Cytokine secretion from the T cells and the CAR-T cells are measured from the cell supernatant after 4 hours co-incubation of cancer cells with the T cells or the CAR-T cells. The result is expected that cytotoxic/cytolytic cytokines are more secreted from and the CAR-T cells upon cancer cell engraftment than the T cells. The cytokine result is anticipated to have correlation with cancer cell death to demonstrate that the increased cancer cell death of the CAR-T cells is induced by the increased cytokine secretion. Consequently, it is anticipated that the recombinant CAR fusion protein can modify T cells to have a CAR system without viral vector-mediated gene transfection and genetic expression time, and consequently, bring the stronger anti-cancer activity.

### Example 12

### (A) Preparation of the Recombinant CAR Fusion Protein Targeting TROP2

A recombinant CAR fusion protein for both NK and T cell targeting TROP2 is designed with mixing the antibody, simultaneously targeting NK and T cell, and intracellular stimulatory region.

The anti-TROP2 CAR fusion protein for both NK and T cells is prepared in the following structure.

Anti-NK&T cell targeting antibody scFv-IgG Hinge-IgG CH2-IgG CH3-linker-Furin cleavage site-membrane targeting-anti-TROP2 scFv-Hinge-Transmembrane region-Intracellular region 1-CD3 zeta-His tag

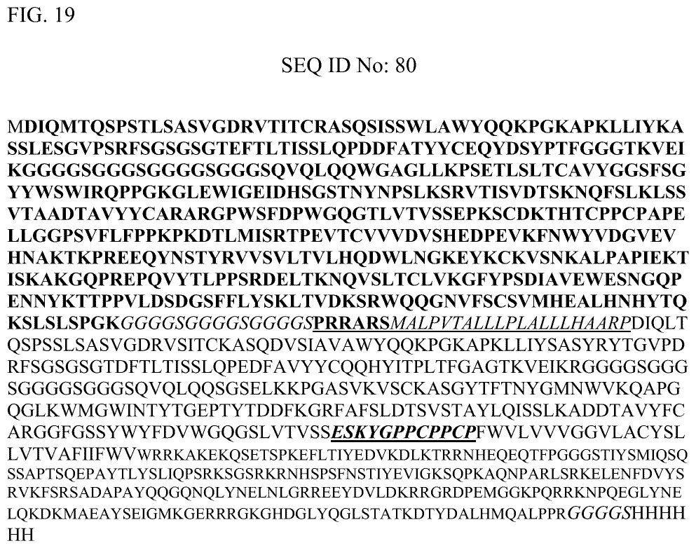

In particular, the amino acid sequence of the recombinant CAR fusion protein targeting TROP2 was as shown in FIG. 19 (SEQ ID NO: 80). The bold letters next to M represent anti-NK&T cell targeting scFv-IgG Hinge-IgG CH2-IgG CH3 (i.e., the immune cell targeting domain). The italicized letters represent a GGGGSGGGGSGGGGS (SEQ ID NO: 79) linker and a GGGGS (SEQ ID NO: 69) linker. The bold underlined letters represent a Furin cleavage site (PRRARS (SEQ ID NO: 80); i.e., the cleavable peptide). The italicized underlined letters represent a membrane targeting domain. The regular capitalized letters represent anti-TROP2 scFv (i.e., the cancer targeting domain). The bold italicized letters represent hinge (i.e., the hinge region). The underlined letters represent transmembrane domain (i.e., the transmembrane domain). The smaller letters represent 2B4-CD3 zeta (i.e., the intracellular signaling domain). HHHHHH (SEQ ID NO: 73) at the C-terminal end is the histidine tag (i.e., the tag sequence).

### (B) Confirmation of the Uptake of the Recombinant CAR Fusion Protein Targeting TROP2 by NK and T Cells

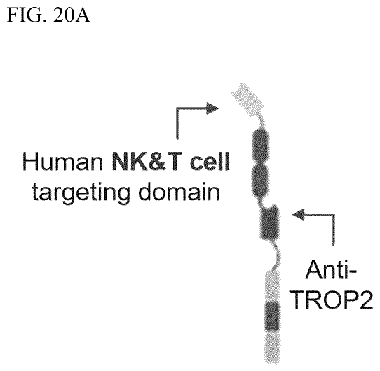

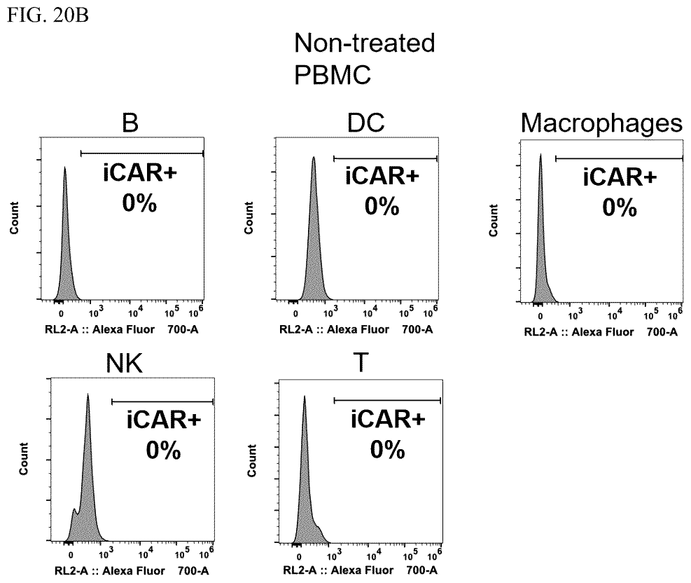

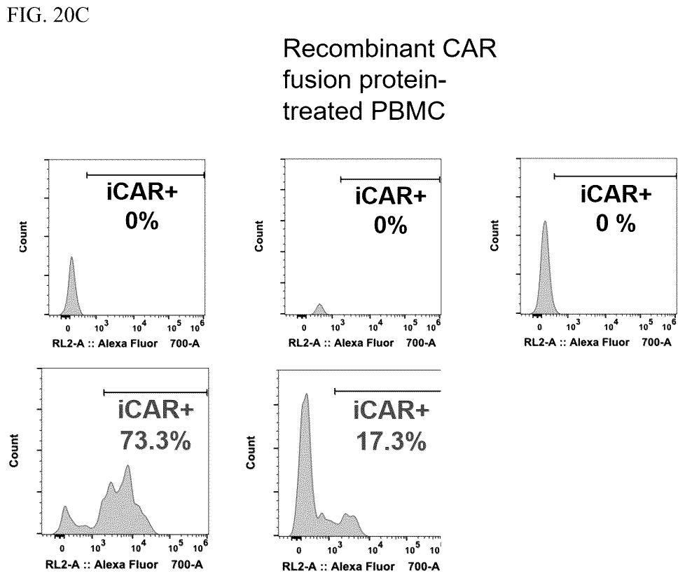

The recombinant CAR fusion protein targeting TROP2 is incubated with human peripheral blood mononuclear cell (PBMC) at 1 μM of the CAR fusion protein (1×10⁶ cells/0.5 mL) for 4-hour. The cells are collected and fixed to be permeabilized. PBMC was labeled with CD68, CD11c, CD19, CD56, and CD3 to distinguish macrophage, B cell, dendritic cell, NK cell and T cell, respectively. Then, Alexa Fluor 700-conjugated anti-his tag antibody was treated to label his-tag on the recombinant CAR fusion protein. As shown in FIGS. 20A, 20B and 20C, the recombinant CAR fusion protein only modified NK and T cells, therebym NK and T cells had high his-tag signal.

### (C) The Recombinant CAR Fusion Protein Transduction Optimization

To optimize concentration for the recombinant CAR fusion protein transduction, the recombinant CAR fusion protein with various concentration is treated to NK and T cells (1×10⁶ cells/0.5 mL) for 24-hour. Protein transduction is confirmed with same method as discussed under Example (B) above. The result is anticipated that the protein uptake is saturated over 1500 nM as like CAR-PD-L1 fusion protein transduction to NK cells, and 1000 nM is selected as the optimal transduction concentration.

### (D) Target (TROP2) Binding Ability of the Recombinant CAR Fusion Protein

After the CAR-NK and CAR-T cells are generated, TROP2 protein is treated to CAR-NK cells, CAR-T cells, NK cells and the T cells and incubated for 2-hour. Because TROP2 protein is biotinylated, the TROP2 protein-bound cells can be identified using APC-streptavidin. The cells are not permeabilized at this experiment in order to stain exterior biotin-tag which is conjugated on TROP2 protein. It is expected that the CAR-NK cells and CAR-T cells have higher APC intensity, which indicates that more TROP2 protein binds to the CAR-NK cells and CAR-T cells than normal NK cells and T cells due to the CAR protein-targeting TROP2.

(E) Cytokine Release from NK Cells, T Cells CAR-NK Cells, or CAR-T Cells after TROP2 Binding Through TROP2 Protein Treatment.

In order to verify the increased anti-cancer activity owing to the CAR fusion protein, cytotoxic/cytolytic cytokine secretion upon TROP2 binding to CAR-NK cells, CAR-T cells, NK cells and T cells is analyzed. After TROP2 binding to the cells (2-hour treatment of 20 ng/mL TROP2 protein), cell supernatant is harvested and ran cytokine ELISA. It is anticipated that cytokine secretions of the CAR-NK cells and CAR-T cells are overly increased as compared to that of the NK cells and T cells. This result implies that the CAR-NK cells and CAR-T cells have stronger anti-cancer activity over normal NK cells and T cells upon the binding with antigen.

### (F) Anti-Cancer Activity of CAR-NK and CAR-T Cell: Cancer Cell Death

Anti-cancer activity of recombinant CAR fusion protein-treated and non-treated PBMC were compared through induction of cancer cell death. The experiment condition is as follows.
CellTracker Blue™ labelled MCF7 cancer cells
Effector:Target (E:T)=3:1
4-hour co-incubation of CellTracker Blue™ labelled-cancer cells with PBMC or recombinant CAR fusion protein-treated PBMC (CAR-PBMC)

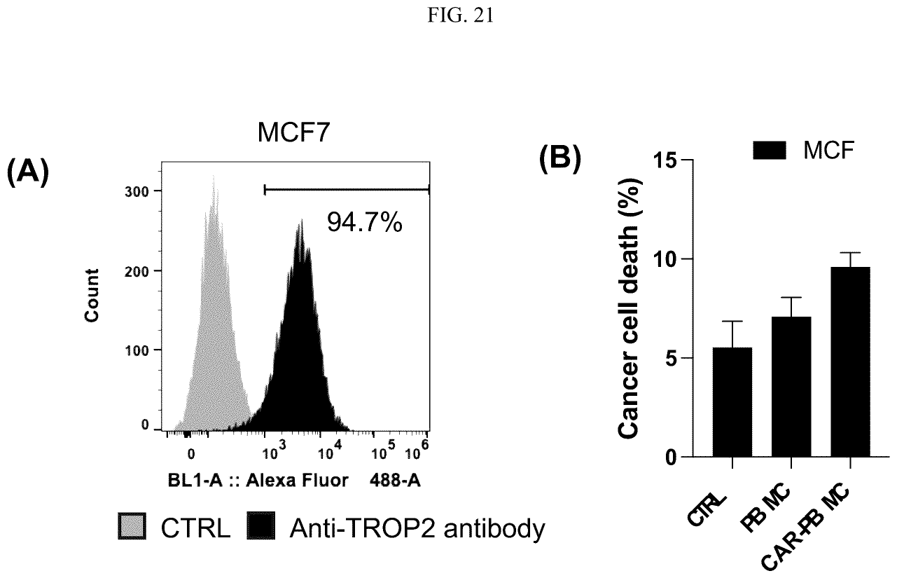

After co-incubation, cells were harvested and stained Alexa Fluor-488 Annexin V to label dead cells. The cells were run through flow cytometry, and the population of CellTracker Blue™ labelled cells (cancer cells) were analyzed the intensity of Alexa Fluor 488. Double positive of CellTracker Blue™ and Alexa Fluor 488-Annexin V-stained cells indicated dead cancer cells. The more population of Alexa Fluor 488-stained cells, the more dead cancer cells. Recombinant CAR fusion protein-treated PBMC induced higher MCF7 cell death compared to PBMC. Along with that, the expression level of TROP2 on MCF7 was validated. MCF7 cells were labeled with Alexa Fluor 488 conjugated anti-mouse IgG antibody for control or Alexa Fluor 488 conjugated anti-TROP2 antibody for test group. The result showed that 94.7% MCF7 cells expressed TROP2. See (A) and (B) of FIG. 21.

### (G) Anti-Cancer Activity of CAR-NK Cells and CAR-T Cell: Cytokine Secretion

The secreted cytokines are measured from the cell supernatant after 4 hours co-incubation of cancer cells with the combination of NK cells and T cells or the other combination of CAR-NK cells and CAR-T cells. The result is expected that cytotoxic/cytolytic cytokines are more secreted from and the combination of CAR-NK cells and CAR-T cells upon cancer cell engraftment than the combination of NK cells and T cells. The cytokine result is anticipated to have correlation with cancer cell death to demonstrate that the increased cancer cell death from the combination group of CAR-NK cells and CAR-T cells is induced by the increased cytokine secretion. Consequently, it is anticipated that the recombinant CAR fusion protein can simultaneously modify NK cells and T cells to have a CAR system without viral vector-mediated gene transfection and genetic expression time, and consequently, bring the stronger anti-cancer activity.

1. A recombinant chimeric antigen receptor (CAR) fusion protein comprising
an immune cell targeting domain,
a cleavable peptide,
a membrane targeting domain,
a cancer targeting domain,
a transmembrane domain, and
an intracellular signaling domain.

2. The recombinant CAR fusion protein of claim 1, wherein the immune cell targeting domain is a NK cell targeting domain, a T cell targeting domain, a dendritic cell targeting domain, a macrophage targeting domain, a peripheral blood mononuclear cell targeting domain, or a B cell targeting domain.

3. The recombinant CAR fusion protein of claim 1, wherein the immune cell targeting domain is an antibody or peptide specifically binding to an immune cell, and comprising one or more of scFv, bispecific scFv, V_{HH}, V_{H}, V_{L}, F_{ab}, CH1, CH2, CH3, or CL.

4. The recombinant CAR fusion protein of claim 1, wherein the cleavable peptide is a peptide which is configured to be enzymatically cleaved.

5. The recombinant CAR fusion protein of claim 1, wherein the membrane targeting domain is a peptide from secretory protein, membrane receptor, surface protein, human oncostatin M, VSV-G, BM40, secrecon, human Ig kappa, human Ig heavy, tPA, human chymotrypsinogen, human trysinogen-2, human IL-2, guassia luciferase, human serum albumin, influenza haemagglutinin, human insulin, silkworm fibroin LC, CD33, CD8, interleukin-1 receptor type 1, 4F2 cell-surface antigen heavy chain, a linker for activation of T-cells family member 1, junctophilin-1, antilisterial bacteriocin subtilosin biosynthesis protein AlbG, calcitonin receptor, gamma-secretase subunit APH-1A, or adipnectin receptor protein 2.

6. The recombinant CAR fusion protein of claim 1, wherein the cancer targeting domain is an antibody or peptide specifically binding to a cancer cell, and comprising one or more of scFv, bispecific scFv, V_{HH}, V_{H}, V_{L}, F_{ab}, CH1, CH2, CH3, or CL.

7. The recombinant CAR fusion protein of claim 1 further comprising a hinge region between the cancer targeting domain and the transmembrane domain.

8. The recombinant CAR fusion protein of claim 1, wherein the transmembrane domain is CD28, CD3, CD4, CD7, CD8, FcεR1γ, ICOS, H2-Kb, NKG2D, CD16, NKp44, or NKp46.

9. The recombinant CAR fusion protein of claim 1, wherein the intracellular signaling domain comprises one or more of CD3 zeta (CD3ζ), 2B4, DAP10, DAP12, GIRT, CD137, OX40, 41BB, CD27, CD28, 2B4, CD137, CD40, and KIR2DS2.

10. The recombinant CAR fusion protein of claim 1 further comprising a tag sequence.

11. The recombinant CAR fusion protein of claim 10, wherein the tag sequence is selected from the group consisting of a metal affinity tag, charge-based tag, epitope peptides, protein-affinity tag, streptavidin/biotin-based tag, histidine tag, glutathione-S-transferase tag, or hemagglutinin tag.

12. A method of modifying an immune cell into a chimeric antigen receptor (CAR) immune cell, comprising
treating the immune cell with the recombinant CAR fusion protein of claim 1.

13. The method of claim 12, wherein the recombinant Car fusion protein is in a concentration of about 100 nM to about 2,000 nM.

14. A method of treating cancer comprising administering the CAR immune cell of claim 12 to a subject in need thereof.

15. A method of treating cancer comprising administering the recombinant CAR fusion protein of claim 1 to a subject in need thereof.

16. The method of claim 15, wherein the recombinant Car fusion protein is in a concentration of about 100 nM to about 2,000 nM.

17. A method of treating cancer comprising administering to a subject in need thereof:
(i) a recombinant chimeric antigen receptor (CAR) fusion protein comprising
an immune cell targeting domain,
a cleavable peptide,
a membrane targeting domain,
a cancer targeting domain,
a transmembrane domain, and
an intracellular signaling domain; and
(ii) a CAR immune cell.

18. The method of claim 17, wherein the CAR immune cell is prepared by treating an immune cell with the recombinant CAR fusion protein.

19. The recombinant CAR fusion protein of claim 1, comprising the sequence of SEQ ID NO: 1.

20. The recombinant CAR fusion protein of claim 1, comprising the sequence of SEQ ID NO: 80.
